(19) United States
(12) Patent Application Publication
(10) Pub. No.: US 2026/0315168 A1
(43) Pub. Date: Oct. 15, 2026

(54) FOLLOW-THE-LEADER (FTL) SMOOTHING FOR A CONTINUUM ROBOT

(71) Applicant: CANON U.S.A., INC., Melville, NY (US)

(72) Inventors: Brian Ninni, Woburn, MA (US); Takahisa Kato, Brookline, MA (US)

(21) Appl. No.: 19/485,986

(22) PCT Filed: May 30, 2024
(86) PCT No.: PCT/US2024/031766

§ 371 Date: Nov. 19, 2025

Related U.S. Application Data
(60) Provisional application No. 63/504,972, filed on May 30, 2023.

(51) Int. Cl. A61B 34/37; A61B 34/00; A61B 34/20; A61B 34/30

(52) U.S. Cl. CPC A61B 34/37; A61B 34/20; A61B 34/71; A61B 2034/2051; A61B 2034/301; A61B 2034/306

(57) ABSTRACT

One or more devices, systems, methods, and storage mediums for performing correction, adjustment, and/or smoothing for a continuum robot are provided herein. Examples of such correction, adjustment, and/or smoothing include, but are not limited to, correction of one or more sections or portions of a continuum robot as the continuum robot is moved, Follow-The-Leader smoothing, state transition(s) or change(s), and path smoothing for a continuum robot. Examples of applications include imaging, evaluating, and diagnosing biological objects, such as, but not limited to, for Gastro-intestinal, cardio, bronchial, and/or ophthalmic applications, and being obtained via one or more optical instruments, such as, but not limited to, optical probes, catheters, endoscopes, and bronchoscopes. Techniques provided herein also improve processing and imaging efficiency while achieving images that are more precise, and also achieve devices, systems, methods, and storage mediums that reduce mental and physical burden and improve ease of use.

### CROSS-REFERENCE TO RELATED APPLICATION(S)

This application relates, and claims priority, to U.S. Prov. Patent Application Ser. No. 63/504,972, filed May 30, 2023, the disclosure of which is incorporated by reference herein in its entirety.

### FIELD OF THE DISCLOSURE

The present disclosure generally relates to imaging and, more particularly, to a continuum robot apparatus, method, and storage medium to implement robotic control for all sections of a catheter or imaging device/apparatus or system to match a state or states when each section reaches or approaches a same or similar, or approximately a same or similar, state or states of a first section of the catheter or imaging device, apparatus, or system. One or more endoscopic, medical, camera, catheter, or imaging devices, systems, and methods and/or storage mediums for use with same, are discussed herein. One or more devices, methods, or storage mediums may be used for medical applications and, more particularly, to steerable, flexible medical devices that may be used for or with guide tools and devices in medical procedures, including, but not limited to, endoscopes, cameras, and catheters.

### BACKGROUND

Endoscopy, bronchoscopy, catheterization, and other medical procedures facilitate the ability to look inside a body. During such a procedure, a flexible medical tool may be inserted into a patient's body, and an instrument may be passed through the tool to examine or treat an area inside the body. For example, a bronchoscope is an endoscopic instrument to view inside the airways of a patient. Catheters and other medical tools may be inserted through a tool channel in the bronchoscope to provide a pathway to a target area in the patient for diagnosis, planning, medical procedure(s), treatment, etc.

Robotic bronchoscopes, robotic endoscopes, or other robotic imaging devices may be equipped with a tool channel or a camera and biopsy tools, and such devices (or users of such devices) may insert/retract the camera and biopsy tools to exchange such components. The robotic bronchoscopes, endoscopes, or other imaging devices may be used in association with a display system and a control system.

An imaging device, such as a camera, may be placed in the bronchoscope, the endoscope, or other imaging device/system to capture images inside the patient and to help control and move the bronchoscope, the endoscope, or the other type of imaging device, and a display or monitor may be used to view the captured images. An endoscopic camera that may be used for control may be positioned at a distal part of a catheter or probe (e.g., at a tip section).

The display system may display, on the monitor, an image or images captured by the camera, and the display system may have a display coordinate used for displaying the captured image or images. In addition, the control system may control a moving direction of the tool channel or the camera. For example, the tool channel or the camera may be bent according to a control by the control system. The control system may have an operational controller (such as, but not limited to, a joystick, a gamepad, a controller, an input device, etc.), and physicians may rotate or otherwise move the camera, probe, catheter, etc. to control same. However, such control methods or systems are limited in effectiveness. Indeed, while information obtained from an endoscopic camera at a distal end or tip section may help decide which way to move the distal end or tip section, such information does not provide details on how the other bending sections or portions of the bronchoscope, endoscope, or other type of imaging device may move to best assist the navigation.

Additionally, in a case where a tip changes orientations or positions while a stage is stationary, the tip change creates a discontinuous path. As such, there is a need to address and improve the discontinuous path.

As such, there is a need for devices, systems, methods, and/or storage mediums that provide the feature(s) or details on how the other bending sections or portions of such imaging devices, imaging systems, etc. (e.g., endoscopic devices, bronchoscopes, other types of imaging devices/systems, etc.) may move to best assist navigation and/or state or state(s) for same, to keep track of a path of a tip of the imaging devices, imaging systems, etc., and to address any discontinuous path that may occur.

Accordingly, it would be desirable to provide at least one imaging, optical, or control device, system, method, and storage medium for controlling one or more endoscopic or imaging devices or systems, for example, by implementing automatic (e.g., robotic) or manual control of each portion or section of the at least one imaging, optical, or control device, system, method, and storage medium to keep track of and to match the state or state(s) of a first portion or section in a case where each portion or section reaches or approaches a same or similar, or approximately same or similar, state or state(s) and to address any discontinuous path that may occur due to movement or a change in a state or state(s).

### SUMMARY

Accordingly, it is a broad object of the present disclosure to provide imaging (e.g., computed tomography (CT), Magnetic Resonance Imaging (MRI), etc.) apparatuses, systems, methods, and storage mediums for using a navigation and/or control method or methods (manual or automatic) in one or more apparatuses or systems (e.g., an imaging apparatus or system, an endoscopic imaging device or system, etc.). The navigation and/or control may be employed so that an apparatus or system having multiple portions or sections (e.g., multiple bending portions or sections) operates to: (i) keep track of a path of a portion (e.g., a tip) or of each of the multiple portions or sections of an apparatus or system; (ii) have a state or states of each of the multiple portions or sections match a state or states of a first portion or section of the multiple portions or sections in a case where each portion or section reaches or approaches a same, similar, or approximately similar state (e.g., a position or other state(s) in a target, object, or specimen; a position or other state(s) in a patient; a target position or state(s) in an image or frame; a set or predetermined position or state(s) in an image or frame; a set or predetermined position or state(s) in an image or frame where the first portion or section reaches or approaches the set or predetermined position or state(s) at one point in time and one or more of other portions or sections of the multiple portions or sections reach the set or predetermined position or state(s) at one or more other points in time; any other state (which may include, but is not limited to, an orientation, a position, a pose, a navigation, a path (whether continuous or discontinuous), a state transition, any other desired motion(s) or combination of motion(s) (e.g., one or more features of the present disclosure may assist navigation, orientation, or any other types of motions discussed herein or as desired by a user), etc.), a combination of any state(s) and/or motion(s) discussed herein or desired by a user, etc.) of another portion or section of the one or more devices, systems, methods, and/or storage mediums of the present disclosure, etc.); and (iii) to address any discontinuous path that may occur (e.g., due to a change (e.g., of a state or states) or other movement or state change/transition of any portion of the apparatus or system), for example, by smoothing out any difference in the discontinuous path over one or more multiple stage positions or states or over one or more other path-like information positions or states. In one or more embodiments, an orientation or state may include one or more degrees of freedom. For example, in at least one orientation embodiment, two (2) degrees of freedom may be used, which may include an angle for a magnitude of bending and a plane for a direction of bending. In one or more embodiments, matching state(s) may involve matching, duplicating, mimicking or otherwise copying other characteristics, such as, but not limited to, vectors for each section or portion of the one or more sections or portions of a probe or catheter, for different portions or sections of the catheter or probe. For example, a transition or change from a base angle/plane to a target angle/plane may be set or predetermined using transition values (e.g., while not limited hereto, a base orientation or state may have a stage at 0 mm, an angle at 0 degrees, and a plane at 0 degrees whereas a target orientation or state may have a stage at 20 mm, an angle at 90 degrees, and a plane at 180 degrees. The intermediate values for the stage, angle, and plane may be set depending on how many transition orientations or states may be used).

One or more embodiments of the present disclosure avoid the aforementioned issues by: (i) providing a simple and fast method or methods that normalizes displaying or viewing images, and/or controlling state(s), navigation, and/or orientation (e.g., rotational orientation, pose, a change in a state or states, etc.), of multiple portions or sections (e.g., multiple bending portions or sections) of an apparatus or system (e.g., a catheter, a probe, an endoscope, a camera, etc.) so that the multiple portions or sections operate to have state(s), a navigation, and/or orientation (or pose, or position, or other state or states discussed herein, etc.) match a navigation and/or orientation (or pose, or position, or state or states transition/change discussed herein, etc.) in a case (or at a time or times) where each portion or section of the multiple portion or sections reaches (or approaches) a same, similar, or approximately same or similar state(s) or position (e.g., a set or predetermined position or state(s), a target position or state(s), a position or state(s) being imaged, another state or states discussed herein (which may include, but are not limited to, an orientation, a position, a pose, a navigation, a path (whether continuous or discontinuous), a state transition, any other desired motion(s) or combination of motion(s) (e.g., one or more features of the present disclosure may assist navigation, orientation, or any other types of motions discussed herein or as desired by a user), etc.), a combination of any state(s) and/or motion(s) discussed herein or desired by a user, etc.) of another portion or section of the one or more devices, systems, methods, and/or storage mediums of the present disclosure, etc.); (ii) keeping track of a path of a portion of the apparatus or system to implement robotic control of a catheter or probe (e.g., by mapping the path or the apparatus or system to a stage position or state or to other path-like information (e.g., a position sensor in a catheter or probe (e.g., an electromagnetic (EM) sensor), another type of sensor, etc.); and/or (iii) having any difference or discrepancy of a discontinuous path smoothed out over one or more multiple stage positions/states or over one or more multiple positions/states for other path-like information or structure being used to map the path-like information. Any discussion of a state, pose, position, orientation, navigation, path, or other state type discussed herein is discussed merely as a non-limiting, non-exhaustive embodiment example, and any state or states discussed herein may be used interchangeably/alternatively or additionally with the specifically mentioned type of state. As such, physicians or other users of the apparatus or system may have reduced or saved labor and/or mental burden using the apparatus or system due to the navigation, control, and/or orientation (or pose, or position, etc.) feature(s) of the present disclosure. Additionally, one or more features of the present disclosure may achieve a minimized or reduced interaction with anatomy (e.g., of a patient), object, or target (e.g., tissue) during use, which may reduce the physical and/or mental burden on a patient or target. In one or more embodiments of the present disclosure, a labor of a user to control and/or navigate (e.g., rotate, translate, etc.) the imaging apparatus or system or a portion thereof (e.g., a catheter, a probe, a camera, one or more sections or portions of a catheter, probe, camera, etc.) is saved or reduced.

In one or more embodiments, an imaging device or system, or a portion of the imaging device or system (e.g., a catheter, a probe, etc.), the multiple sections or portions may be multiple bending sections or portions. In one or more embodiments the imaging device or system may include manual and/or automatic navigation and/or control features. For example, a user of the imaging device or system may control each section or portion, and/or the imaging device or system may operate to automatically control (e.g., robotically control) each section or portion.

Navigation, control, and/or orientation feature(s) may include, but are not limited to, implementing mapping of a pose (angle value(s), plane value(s), etc.) of a first portion or section (e.g., a tip portion or section, a distal portion or section, a predetermined or set portion or section, a user selected or defined portion or section, etc.) to a stage position/state (or a position/state of another structure being used to map path or path-like information), controlling angular position(s) of one or more of the multiple portions or sections, controlling rotational orientation or position(s) of one or more of the multiple portions or sections, controlling (manually or automatically (e.g., robotically)) one or more other portions or sections of the imaging device or system to match the navigation/orientation/position/pose of the first portion or section in a case where the one or more other portions or sections reach (e.g., subsequently reach, reach at a different time, etc.) the same position (e.g., in a target, in an object, in a sample, in a patient, in a frame or image, etc.) during navigation in or along a first direction of a path of the imaging device or system, controlling each of the sections or portions of the imaging device or system to retrace and match prior respective position(s) of the sections or portions in a case where the imaging device or system is moving or navigated in a second direction (e.g., in an opposite direction along the path, in a return direction along the path, in a retraction direction along the path, etc.) along the path, etc. For example, an imaging device or system (or portion thereof, such as, but not limited to, a probe, a catheter, a camera, etc.) may enter a target along a path where a first section or portion of the imaging device or system (or portion of the device or system) is used to set the navigation or control path and position(s), and each subsequent section or portion of the imaging device or system (or portion of the device or system) is controlled to follow the first section or portion such that each subsequent section or portion matches the orientation and position of the first section or portion at each location along the path. During retraction, each section or portion of the imaging device or system is controlled to match the prior orientation and position (for each section or portion) for each of the locations along the path. As such, an imaging device or system (or catheter, probe, camera, etc. of the device or system) may enter and exit a target, an object, a specimen, a patient (e.g., a lung of a patient, an esophagus of a patient, another portion of a patient, another organ of a patient, a vessel of a patient, etc.), etc. along the same path and using the same orientation for entrance and exit to achieve an optimal navigation, orientation, and/or control path. The navigation, control, and/or orientation feature(s) are not limited thereto, and one or more devices or systems of the present disclosure may include any other desired navigation, control, and/or orientation specifications or details as desired for a given application or use. In one or more embodiments and while not limited thereto, the first portion or section may be a distal or tip portion or section of the imaging device or system. In one or more embodiments, the first portion or section may be any predetermined or set portion or section of the imaging device or system, and the first portion or section may be predetermined or set manually by a user of the imaging device or system or may be set automatically by the imaging device or system.

In one or more embodiments, an effective manner of smoothing may be to aim a section (e.g., a distal or tip portion or section) in a direct path between orientations. In one or more embodiments, the direct path may be the shortest path and may use a smallest possible volume for efficiency. One or more embodiments may use indirect path(s). In a case where an indirect path may be used, a motion (e.g., of one section, of a first portion or section, etc.) may also affect subsequent portions or sections of the catheter or probe. In a case where a direct path may be used, the motion and any resulting effect on subsequent portions or sections is reduced and/or minimized. Such a reduction or minimization is important and useful for a user of the catheter or probe or is useful for a continuum robot system or apparatus that is automatically controlling the catheter or probe in a case where a camera is at the very end or Tip portion or section, or close to an end or Tip portion or section, of the catheter or probe. To reduce and/or avoid an undesired motion (e.g., that may be caused by an indirection motion that may affect the Tip section or other portion of the catheter or probe), a user may adjust the Tip portion or section orientation (e.g., manually), or the system or apparatus may operate (e.g., via one or more processors thereof) to adjust the Tip portion or section orientation automatically. In one or more embodiments, as a user moves a device or system forward (or a portion of the device or system forward, such as, but not limited to, a stage, a structure used for mapping a path or path-like information, a probe, a catheter, etc.), an imaging view or a camera view may also move forward. In one or more embodiments, in a case where an indirect path approaches a final orientation, the orientation of the catheter or probe moving along the indirect path may use an adjustment (e.g., the adjustment to the Tip portion or section orientation). An additional adjustment may be used in a case where the orientation adjustment (e.g., the adjustment to the Tip portion or section orientation) may be undone or removed by the additional adjustment (e.g., in a case where the original or initial orientation adjustment may have been avoided or may have been unnecessary, etc.). Since the orientations may be stored in history (e.g., in a memory or other storage medium such as, but not limited to, the memories or storage mediums discussed herein, memories known to those skilled in the art, etc.), the orientations may be repeated by each of the following sections (e.g., each section after the Tip section or portion), and adjustments may be performed by a user (e.g., manually) or via one or more processors of the apparatus or system (e.g., automatically) to account for, or make adjustments or corrections to, unnecessary motion or motion that may be made more efficient. In one or more embodiments, the adjustments may be stored in history along with the orientations such that the system or apparatus may perform the adjustments to each of the following sections such that a user may avoid having to repeatedly account for unnecessary motion manually because the system or apparatus performs or provides this feature.

One or more embodiments of the present disclosure may implement a direct path smoothing by having each stage position/state information (or other structure-based path or path-like state/position information) along a smoothing range gradually aim towards a final orientation. To make each stage position/state (or other path-like position/state) along a smoothing range gradually aim towards a final orientation, a system or apparatus may have a “change in orientation” that occurs at a single stage position/state (or other structure-based mapping path-like position/state) be interpolated to or between steps, with each step being closer to the final orientation than the previous step.

In one or more embodiments of the present disclosure (and while not limited to only this definition), a “change of orientation” may be defined in terms of direction and magnitude. For example, each interpolated step may have a same direction, and each interpolated step may have a larger magnitude as each step approaches a final orientation. Due to kinematics of one or more embodiments, any motion along a single direction may be the accumulation of a small motion in that direction. The small motion may have a unique or predetermined set of wire position changes to achieve the orientation change. Large or larger motion(s) in that direction may use a plurality of the small motions to achieve the large or larger motion(s). Dividing a large change into a series of multiple changes of the small or predetermined/set change may be used as one way to perform interpolation. Interpolation may be used in one or more embodiments to produce a desired or target motion, and at least one way to produce the desired or target motion may be to interpolate the change of wire positions.

In one or more embodiments, where smoothing is performed, interpolated orientations may be combined with values previously mapped to a corresponding stage position/state (or position/state of another structure used for mapping path or path-like information) (e.g., by simply adding the wire positions). Three points of drive or driving wire positions/states operate to define a plane, and two poses may have specific drive or driving wire positions or states. Interpolation may be performed between the two positions or states for a transition (e.g., a direct transition, a direct orientation transition, a direct position transition, a constant relative orientation transition, etc.).

Due to kinematics of a robot, device, or system embodiment of the present disclosure, applying a same “change in position” to two separate orientations may maintain a difference (e.g., a set difference, a predetermined difference, etc.) between the two separate orientations. Since an orientation difference may be defined as the difference between wire position in one or more embodiments (other embodiments are not limited thereto), changing both sets of wire positions by the same amount would not affect the orientation difference between the two separate orientations. In one or more embodiments, drive or driving wires (or other structural actuators being used) may be in positions or states that do not match kinematic model(s), and adjustments may or may not be used (as desired) to return the position or state to match the kinematic model(s) (such adjustments may be omitted in one or more embodiments).

Orientations mapped to two subsequent stage positions/states (or positions/states of another structure used for mapping path or path-like information) may have a specific orientation/state difference between the orientations/states. In a case where smoothing is applied, the later (or second) stage position/state (or position/state of another structure used for mapping the path or path-like information) has a same change in orientation/state that the earlier (or first) stage position/state (or position/state of another structure used for mapping the path or path-like information) received such that the pose/state difference did not change. The smoothing process may include an additional step of a “small motion”, which operates to cause the pose/state difference to change by an amount of that small motion or state change. Since the “small motion” operates to produce the same orientation or state change regardless of prior orientation or state, the small motion step operates to direct that orientation in a table towards a proper (e.g., set, desired, predetermined, selected, etc.) direction, while also maintaining a semblance or configuration of the prior path before the smoothing process was applied. Therefore, in one or more embodiments, it may be most efficient and effective to combine and compare wire positions to or with prior orientations while using a smoothing process to maintain the pre-existing orientation/state changes.

In one or more embodiments, a catheter or probe may transition, move, or adjust using a shortest possible volume. In a case where a following section or portion of the probe or catheter is being transitioned, moved, or adjusted, using the shortest possible volume my reduce or minimize an amount of disruption to positions of one or more (or all) of the distal/following sections or portions of the catheter or probe. In one or more embodiments, a process or algorithm may perform the transitioning, moving, or adjusting process more efficiently than computing a transformation stackup of each section or portion of the catheter or probe. Preferably, each interpolated step aims towards the final orientation in a desired direction such that any prior orientation which the interpolated step is combined with will also aim towards the desired direction to achieve the final orientation.

In one or more embodiments of the present disclosure, an apparatus or system may include one or more processors that operate to: instruct or command a distal bending section or portion of a catheter or a probe of the continuum robot such that the distal bending section or portion achieves, or is disposed at, a bending pose or position, the catheter or probe of the continuum robot having a plurality of bending sections or portions and a base; store or obtain the bending pose or position of the distal bending section or portion and store or obtain a position or state of a motorized linear stage (or other structure used to map path or path-like information) that operates to move the catheter or probe of the continuum robot in a case where the one or more processors instruct or command forward motion, or a motion in a set or predetermined direction or directions, of the motorized linear stage (or other predetermined or set structure for mapping path or path-like information); generate a goal or target bending pose or position for each corresponding section or portion of the catheter or probe from, or based on, the previous bending section or portion; generate interpolated poses or positions for each of the sections or portions of the catheter or probe between the respective goal or target bending pose or position and a respective current bending pose or position of each of the sections or portions of the catheter or probe, wherein the interpolated poses or positions are generated such that an orientation vector of the interpolated poses or positions are on a plane that an orientation vector of the respective goal or target bending pose or position and an orientation vector of a respective current bending pose or position create or define; and instruct or command each of the sections or portions of the catheter or probe to move to or be disposed at the respective interpolated poses or positions during the forward motion, or the motion in the set or predetermined direction, of the previous section(s) or portion(s) of the catheter or probe.

In one or more embodiments, an apparatus/device or system may have one or more of the following exist or occur: (i) the distal bending section or portion may be the most distal bending section or portion, and the most distal bending section or portion may be commanded or instructed automatically or based on an input of a user of the continuum robot in a case where the motorized linear stage (or other structure used for mapping path or path-like information) is stable or stationary; (ii) the plurality of bending sections or portions may include the distal or most distal bending portion or section and the rest of the plurality of the bending sections or portions; (iii) the one or more processors may further operate to instruct or command the forward motion, or the motion in the set or predetermined direction, of the motorized linear stage (or other structure used for mapping path or path-like information) automatically or based on an input of a user of the continuum robot; and/or (iv) the plane may be created or defined based on a base coordinate system or based on a system substantially close to the base coordinate system.

In one or more embodiments, an apparatus or system (e.g., of or including a continuum robot) may further include: an actuator that operates to bend the plurality of the bending sections or portions independently and the base; and the motorized linear stage (or other structure used for mapping path or path-like information) that operates to move the continuum robot forward and backward, and/or in the predetermined or set direction or directions, wherein the one or more processors operate to control the actuator and the motorized linear stage (or other structure used for mapping path or path-like information). One or more embodiments may include a user interface of or disposed on the base, or disposed remotely from the base, the user interface operating to receive an input from a user of the continuum robot to move one or more of the plurality of bending sections or portions and/or the motorized linear stage (or other structure used for mapping path or path-like information), wherein the one or more processors further operate to receive the input from the user interface, and the one or more processors and/or the user interface operate to use a base coordinate system.

In one or more embodiments, the plurality of bending sections or portions may each include drive or driving wires that operate to bend a respective section or portion of the plurality of sections or portions, wherein the drive or driving wires are connected to the actuator so that the actuator operates to bend the plurality of bending sections or portions using the drive or driving wires. The one or more processors may further operate to one or more of the following: generate the interpolated poses or positions by interpolating positions of the drive or driving wires between the respective goal or target positions and the respective current positions; take a difference in drive or driving wire position between a value stored in a table and a new value; and disperse an adjustment, correction, and/or smoothing along an entire length of travel from a current position of the bending sections or portions by: (i) calculating a number of steps by multiplying a length of a section or portion and a table resolution; (ii) interpolating the value from the table into equal portions where the number of the equal portions equals the calculated number of steps, starting with the total value and ending at zero; and (iii) working backwards from the position in the table where the new value is getting stored, and adding the interpolated position value to the corresponding value stored in the table, wherein StagePosition[0] (or Position[0] of another structure used for mapping path or path-like information) is the position where the new value is to be stored, InterpolatedPosition[0] is the full amount of the wire position difference, StagePosition[−i] (or Position[−i] of the other structure used for mapping path or path-like information) is the position where the smoothing beings, and InterpolatedPosition[i] has zero wire position difference.

In one or more embodiments, the adjustment, correction, and/or smoothing may occur such that any intermediate orientations of one or more of the plurality of bending sections or portions is guided towards respective desired, predetermined, or set orientations.

In one or more embodiments, the catheter or probe of the continuum robot may be a steerable catheter or probe including the plurality of bending sections or portions and including an endoscope camera, wherein the one or more processors further operate to receive one or more endoscopic images from the endoscope camera, and wherein the continuum robot further comprises a display that operates to display the one or more endoscopic images.

One or more embodiments may include one or more of the following features: (i) the continuum robot may further include an operational controller or joystick that operates to issue or input one or more commands or instructions as an input to the one or more processors, the input including an instruction or command to move one or more of the plurality of bending sections or portions and/or the motorized linear stage (or other structure used for mapping path or path-like information); (ii) the continuum robot may further include a display to display one or more images taken by the continuum robot; and/or (iii) the continuum robot may further include an operational controller or joystick that operates to issue or input one or more commands or instructions to the one or more processors, the input including an instruction or command to move one or more of the plurality of bending sections or portions and/or the motorized linear stage (or other structure used for mapping path or path-like information), and the operational controller or joystick operates to be controlled by a user of the continuum robot.

In one or more embodiments of the present disclosure, an apparatus or system may include one or more processors that operate to: receive or obtain an image or images showing pose or position information of a tip section of a catheter or probe having a plurality of sections including at least the tip section; track a history of the pose or position information of the tip section of the catheter or probe during a period of time; and use the history of the pose or position information of the tip section to determine how to align or transition, move, or adjust (e.g., robotically, manually, automatically, etc.) each section of the plurality of sections of the catheter or probe.

In one or more embodiments, one or more additional image or images may be received or obtained to show the catheter or probe after each section of the plurality of sections of the catheter or probe has been aligned or adjusted (e.g., robotically, manually, automatically, etc.) based on the history of the pose or position information of the tip section. In one or more embodiments, the apparatus or system may include a display to display the image or images showing the aligned or adjusted sections of the catheter or probe. In one or more embodiments, the pose or position information may include, but is not limited to, a target pose or position or a final pose or position that the tip section is set to reach, an interpolated pose or position of the tip section (e.g., an interpolation of the tip section between two positions or poses (e.g., between pose or position A to pose or position B) where the apparatus or system sends pose change information in steps based on a desired, set, or predetermined speed; between poses or positions where each pose or position of the catheter or probe takes or is disposed is tracked during the transition; etc.), and a measured pose or position (e.g., using tracked poses or positions, using encoder positions of each wire motor, etc.) where the one or more processors may further operate to calculate or derive a current position or position that a section (e.g., the tip section, one of the other sections of the plurality of sections of the probe or catheter, etc.) of the probe or catheter is taking. In addition to using one or more types of poses or positions, each pose or position may be converted (e.g., via the one or more processors) between the following formats: Drive/Driving Wire Positions; and/or Coordinates (three-dimensional (3D) Position and Orientation).

In one or more embodiments, an apparatus or system may include a camera deployed at a tip of a catheter or probe and may be bent with the catheter or probe, and/or the camera may be detachably attached to, or removably inserted into, the steerable catheter or probe. In one or more embodiments, an apparatus or system may include a display controller, or the one or more processors may display the image or images for display on a display. One or more embodiments may further include the display to display the adjusted, corrected, or smoothed path of the continuum robot.

In one or more embodiments, a method for performing correction, adjustment, and/or for smoothing (e.g., FTL smoothing, path smoothing etc.) may include: instructing or commanding a distal bending section or portion of a catheter or a probe of the continuum robot such that the distal bending section or portion achieves, or is disposed at, a bending pose or position, the catheter or probe of the continuum robot having a plurality of bending sections or portions and a base; storing or obtaining the bending pose or position of the distal bending section or portion and storing or obtaining a position of a motorized linear stage (or other structure used for mapping path or path-like information) that operates to move the catheter or probe of the continuum robot in a case where a forward motion, or a motion in a set or predetermined direction or directions, of the motorized linear stage (or other structure used for mapping path or path-like information) is instructed or commanded; generating a goal or target bending pose or position for each corresponding section or portion of the catheter or probe from, or based on, the previous bending section or portion; generating interpolated poses or positions for each of the sections or portions of the catheter or probe between the respective goal or target bending pose or position and a respective current bending pose or position of each of the sections or portions of the catheter or probe, wherein the interpolated poses or positions are generated such that an orientation vector of the interpolated poses or positions are on a plane that an orientation vector of the respective goal or target bending pose or position and an orientation vector of a respective current bending pose or position create or define; and instructing or commanding each of the sections or portions of the catheter or probe to move to or be disposed at the respective interpolated poses or positions during the forward motion, or the motion in the set or predetermined direction, of the previous section(s) or portion(s) of the catheter or probe.

In one or more embodiments, a non-transitory computer-readable storage medium may store at least one program for causing a computer to execute a method for performing correction, adjustment, smoothing (e.g., FTL smoothing, path smoothing, etc.), the method comprising: instructing or commanding a distal bending section or portion of a catheter or a probe of the continuum robot such that the distal bending section or portion achieves, or is disposed at, a bending pose or position, the catheter or probe of the continuum robot having a plurality of bending sections or portions and a base; storing or obtaining the bending pose or position of the distal bending section or portion and storing or obtaining a position of a motorized linear stage (or other structure used for mapping path or path-like information) that operates to move the catheter or probe of the continuum robot in a case where a forward motion, or a motion in a set or predetermined direction or directions, of the motorized linear stage (or other structure used for mapping path or path-like information) is instructed or commanded; generating a goal or target bending pose or position for each corresponding section or portion of the catheter or probe from, or based on, the previous bending section or portion; generating interpolated poses or positions for each of the sections or portions of the catheter or probe between the respective goal or target bending pose or position and a respective current bending pose or position of each of the sections or portions of the catheter or probe, wherein the interpolated poses or positions are generated such that an orientation vector of the interpolated poses or positions are on a plane that an orientation vector of the respective goal or target bending pose or position and an orientation vector of a respective current bending pose or position create or define; and instructing or commanding each of the sections or portions of the catheter or probe to move to or be disposed at the respective interpolated poses or positions during the forward motion, or the motion in the set or predetermined direction, of the previous section(s) or portion(s) of the catheter or probe.

In one or more embodiments, a continuum robot for performing correction, adjustment, and/or smoothing, may include: one or more processors that operate to: instruct or command a distal bending section or portion of a catheter or a probe of the continuum robot such that the distal bending section or portion achieves, or is disposed at, a bending pose, position, or state, the catheter or probe of the continuum robot having a plurality of bending sections or portions and a base; store or obtain the bending pose, position, or state of the distal bending section or portion and store or obtain a position or state of a motorized linear stage and/or of a sensor that operates to move the catheter or probe of the continuum robot in a case where the one or more processors instruct or command forward motion, or a motion in a set or predetermined direction or directions, of the motorized linear stage and/or of the sensor; generate a goal or target bending pose, position, or state for each corresponding section or portion of the catheter or probe from, or based on, the previous bending section or portion; determine interpolated poses, positions, or states for each corresponding section or portion of the catheter or probe based on adjacent interpolated poses, positions or states such that all interpolated poses, positions, or states include the same or similar displacement vector between the adjacent interpolated poses, positions, or states based on an end-effector coordinate; and instruct or command each of the sections or portions of the catheter or probe to move to or be disposed at the respective interpolated poses, positions, or states during the forward motion, or the motion in the set or predetermined direction, of the previous section(s) or portion(s) of the catheter or probe. In one or more embodiments, the same or similar displacement vector between the adjacent interpolated poses, positions, or states may be based on the end-effector coordinate from a view point of a distal end of that respective section or portion of the catheter or probe. In one or more embodiments, the one or more processors may further operate to one or more of the following: generate the interpolated poses, positions, or states by interpolating positions or states of the drive or driving wires between the respective goal or target poses, positions, or states and the respective current poses, positions, or states; take a difference in drive or driving wire position between a value stored in a table and a new value; and disperse an adjustment, correction, and/or smoothing along an entire length of travel from a current pose, position, or state of the bending sections or portions by: (i) calculating a number of steps by multiplying a length of a section or portion and a table resolution; (ii) interpolating the value from the table into equal portions where the number of the equal portions equals the calculated number of steps, starting with the total value and ending at zero; and (iii) working backwards from the position in the table where the new value is getting stored, and adding the interpolated pose, position, or state value to the corresponding value stored in the table, wherein StagePosition[0] is the position where the new value is to be stored, InterpolatedPosition[0] is the full amount of the wire pose, position, or state difference, StagePosition[−i] is the pose, position, or state where the smoothing begins, and InterpolatedPosition[i] has zero wire pose, position, or state difference.

In one or more embodiments, a method for performing correction, adjustment, and/or smoothing for a continuum robot may include: instructing or commanding a distal bending section or portion of a catheter or a probe of the continuum robot such that the distal bending section or portion achieves, or is disposed at, a bending pose, position, or state, the catheter or probe of the continuum robot having a plurality of bending sections or portions and a base; storing or obtaining the bending pose, position, or state of the distal bending section or portion and store or obtain a position or state of a motorized linear stage and/or of a sensor that operates to move the catheter or probe of the continuum robot in a case where the one or more processors instruct or command forward motion, or a motion in a set or predetermined direction or directions, of the motorized linear stage and/or of the sensor; generating a goal or target bending pose, position, or state for each corresponding section or portion of the catheter or probe from, or based on, the previous bending section or portion; determining interpolated poses, positions, or states for each corresponding section or portion of the catheter or probe based on adjacent interpolated poses, positions or states such that all interpolated poses, positions, or states include the same or similar displacement vector between the adjacent interpolated poses, positions, or states based on an end-effector coordinate; and instructing or commanding each of the sections or portions of the catheter or probe to move to or be disposed at the respective interpolated poses, positions, or states during the forward motion, or the motion in the set or predetermined direction, of the previous section(s) or portion(s) of the catheter or probe.

In one or more embodiments, a non-transitory computer-readable storage medium may store at least one program for causing a computer to execute a method for performing correction, adjustment, and/or smoothing for a continuum robot, where the method may include: instructing or commanding a distal bending section or portion of a catheter or a probe of the continuum robot such that the distal bending section or portion achieves, or is disposed at, a bending pose, position, or state, the catheter or probe of the continuum robot having a plurality of bending sections or portions and a base; storing or obtaining the bending pose, position, or state of the distal bending section or portion and store or obtain a position or state of a motorized linear stage and/or of a sensor that operates to move the catheter or probe of the continuum robot in a case where the one or more processors instruct or command forward motion, or a motion in a set or predetermined direction or directions, of the motorized linear stage and/or of the sensor; generating a goal or target bending pose, position, or state for each corresponding section or portion of the catheter or probe from, or based on, the previous bending section or portion; determining interpolated poses, positions, or states for each corresponding section or portion of the catheter or probe based on adjacent interpolated poses, positions or states such that all interpolated poses, positions, or states include the same or similar displacement vector between the adjacent interpolated poses, positions, or states based on an end-effector coordinate; and instructing or commanding each of the sections or portions of the catheter or probe to move to or be disposed at the respective interpolated poses, positions, or states during the forward motion, or the motion in the set or predetermined direction, of the previous section(s) or portion(s) of the catheter or probe.

In one or more embodiments, a continuum robot for performing correction, adjustment, and/or smoothing, may include: one or more processors that operate to: instruct or command a distal bending section or portion of a catheter or a probe of the continuum robot such that the distal bending section or portion achieves, or is disposed at, a bending pose, position, or state, the catheter or probe of the continuum robot having a plurality of bending sections or portions and a base; store or obtain the bending pose, position, or state of the distal bending section or portion and store or obtain a position or state of a motorized linear stage and/or of a sensor that operates to move the catheter or probe of the continuum robot in a case where the one or more processors instruct or command forward motion, or a motion in a set or predetermined direction or directions, of the motorized linear stage and/or of the sensor; generate a goal or target bending pose, position, or state for each corresponding section or portion of the catheter or probe from, or based on, the previous bending section or portion; determine interpolated poses, positions, or states for each corresponding section or portion of the catheter or probe based on a pose, position, or state of a tip section or portion of the catheter or probe defining a start pose, position, or state and based on a target or goal pose, position, or state for each corresponding section or portion of the catheter or probe; and instruct or command each of the sections or portions of the catheter or probe to move to or be disposed at the respective interpolated poses, positions, or states during the forward motion, or the motion in the set or predetermined direction, of the previous section(s) or portion(s) of the catheter or probe. In one or more embodiments, the one or more processors may further operate to determine the determined interpolated poses, positions, or states based on: (i) a plane of the start pose, position, or state and the goal or target pose, position, or state; and/or (ii) an origin of a base coordinate.

In one or more embodiments, a method for performing correction, adjustment, and/or smoothing for a continuum robot may include: instructing or commanding a distal bending section or portion of a catheter or a probe of the continuum robot such that the distal bending section or portion achieves, or is disposed at, a bending pose, position, or state, the catheter or probe of the continuum robot having a plurality of bending sections or portions and a base; storing or obtaining the bending pose, position, or state of the distal bending section or portion and store or obtain a position or state of a motorized linear stage and/or of a sensor that operates to move the catheter or probe of the continuum robot in a case where the one or more processors instruct or command forward motion, or a motion in a set or predetermined direction or directions, of the motorized linear stage and/or of the sensor; generating a goal or target bending pose, position, or state for each corresponding section or portion of the catheter or probe from, or based on, the previous bending section or portion; determining interpolated poses, positions, or states for each corresponding section or portion of the catheter or probe based on a pose, position, or state of a tip section or portion of the catheter or probe defining a start pose, position, or state and based on a target or goal pose, position, or state for each corresponding section or portion of the catheter or probe; and instructing or commanding each of the sections or portions of the catheter or probe to move to or be disposed at the respective interpolated poses, positions, or states during the forward motion, or the motion in the set or predetermined direction, of the previous section(s) or portion(s) of the catheter or probe.

In one or more embodiments, a non-transitory computer-readable storage medium may store at least one program for causing a computer to execute a method for performing correction, adjustment, and/or smoothing for a continuum robot, where the method may include: instructing or commanding a distal bending section or portion of a catheter or a probe of the continuum robot such that the distal bending section or portion achieves, or is disposed at, a bending pose, position, or state, the catheter or probe of the continuum robot having a plurality of bending sections or portions and a base; storing or obtaining the bending pose, position, or state of the distal bending section or portion and store or obtain a position or state of a motorized linear stage and/or of a sensor that operates to move the catheter or probe of the continuum robot in a case where the one or more processors instruct or command forward motion, or a motion in a set or predetermined direction or directions, of the motorized linear stage and/or of the sensor; generating a goal or target bending pose, position, or state for each corresponding section or portion of the catheter or probe from, or based on, the previous bending section or portion; determining interpolated poses, positions, or states for each corresponding section or portion of the catheter or probe based on a pose, position, or state of a tip section or portion of the catheter or probe defining a start pose, position, or state and based on a target or goal pose, position, or state for each corresponding section or portion of the catheter or probe; and instructing or commanding each of the sections or portions of the catheter or probe to move to or be disposed at the respective interpolated poses, positions, or states during the forward motion, or the motion in the set or predetermined direction, of the previous section(s) or portion(s) of the catheter or probe.

In accordance with one or more embodiments of the present disclosure, apparatuses and systems, and methods and storage mediums for performing correction(s) and/or adjustment(s) to a direction or view, and/or for performing smoothing (e.g., direct FTL smoothing, path smoothing for a continuum robot, etc.), may operate to characterize biological objects, such as, but not limited to, blood, mucus, tissue, etc.

One or more embodiments of the present disclosure may be used in clinical application(s), such as, but not limited to, intervascular imaging, intravascular imaging, bronchoscopy, atherosclerotic plaque assessment, cardiac stent evaluation, intracoronary imaging using blood clearing, balloon sinuplasty, sinus stenting, arthroscopy, ophthalmology, ear research, veterinary use and research, etc.

In accordance with at least another aspect of the present disclosure, one or more technique(s) discussed herein may be employed as or along with features to reduce the cost of at least one of manufacture and maintenance of the one or more apparatuses, devices, systems, and storage mediums by reducing or minimizing a number of optical and/or processing components and by virtue of the efficient techniques to cut down cost (e.g., physical labor, mental burden, fiscal cost, time and complexity, etc.) of use/manufacture of such apparatuses, devices, systems, and storage mediums.

The following paragraphs describe certain explanatory embodiments. Other embodiments may include alternatives, equivalents, and modifications. Additionally, the explanatory embodiments may include several novel features, and a particular feature may not be essential to some embodiments of the devices, systems, and methods that are described herein.

According to other aspects of the present disclosure, one or more additional devices, one or more systems, one or more methods, and one or more storage mediums using imaging adjustment or correction and/or other technique(s) are discussed herein. Further features of the present disclosure will in part be understandable and will in part be apparent from the following description and with reference to the attached drawings.

### BRIEF DESCRIPTION OF THE DRAWINGS

For the purposes of illustrating various aspects of the disclosure, wherein like numerals indicate like elements, there are shown in the drawings simplified forms that may be employed, it being understood, however, that the disclosure is not limited by or to the precise arrangements and instrumentalities shown. To assist those of ordinary skill in the relevant art in making and using the subject matter hereof, reference is made to the appended drawings and figures, wherein:

FIG. 1 illustrates at least one embodiment of an imaging, continuum robot, or endoscopic apparatus or system in accordance with one or more aspects of the present disclosure;

FIG. 2 is a schematic diagram showing at least one embodiment of an imaging, continuum robot, or endoscopic apparatus or system in accordance with one or more aspects of the present disclosure;

FIG. 3 is a schematic diagram showing at least one embodiment of a console or computer that may be used with one or more correction, adjustment, or smoothing technique(s) in accordance with one or more aspects of the present disclosure;

FIGS. 4A-4B illustrate at least one embodiment example of a continuum robot and/or medical device that may be used with one or more correction, adjustment, or smoothing technique(s) in accordance with one or more aspects of the present disclosure;

FIG. 5 is a schematic diagram showing at least one embodiment of an imaging or continuum robot apparatus or system in accordance with one or more aspects of the present disclosure;

FIG. 6 is a flowchart of at least one embodiment of a method for planning an operation of at least one embodiment of a continuum robot apparatus or system in accordance with one or more aspects of the present disclosure;

FIG. 7 illustrates a graph and related information of at least one embodiment of a method for performing one or more correction, adjustment, or smoothing technique(s) of at least one embodiment of a continuum robot apparatus or system in accordance with one or more aspects of the present disclosure;

FIG. 8 illustrates a graph and related information of the at least one embodiment of FIG. 7 for performing one or more correction, adjustment, or smoothing technique(s), as compared with an indirect path or technique, of at least one embodiment of a continuum robot apparatus or system in accordance with one or more aspects of the present disclosure;

FIG. 9 illustrates a graph and related information of at least one additional embodiment of a method for performing one or more correction, adjustment, or smoothing technique(s) of at least one embodiment of a continuum robot apparatus or system in accordance with one or more aspects of the present disclosure;

FIGS. 10A-10C show three (top) orientation vector trace graphs for isometric, side, and top information, respectively, and show three (bottom) end coordinates trace graphs for isometric, side, and top information, respectively, for different paths in accordance with one or more aspects of the present disclosure;

FIGS. 11A-11C show three (top) orientation vector trace graphs for isometric, side, and top information, respectively, and show three (bottom) end coordinates trace graphs for isometric, side, and top information, respectively, for a same or similar path in accordance with one or more aspects of the present disclosure;

FIG. 12 is a diagram illustrating features of one or more correction, adjustment, or smoothing techniques and/or structures of at least one embodiment of a continuum robot apparatus or system in accordance with one or more aspects of the present disclosure;

FIG. 13 is a diagram illustrating features of one or more additional correction, adjustment, or smoothing techniques and/or structures of at least one embodiment of a continuum robot apparatus or system in accordance with one or more aspects of the present disclosure;

FIG. 14 is a diagram illustrating features of one or more further correction, adjustment, or smoothing techniques and/or structures of at least one embodiment of a continuum robot apparatus or system in accordance with one or more aspects of the present disclosure;

FIG. 15 is a flowchart of at least one embodiment of a method for performing adjustment, correction, or smoothing for a continuum robot in accordance with one or more aspects of the present disclosure;

FIG. 16 illustrates a diagram of a continuum robot that may be used with one or more adjustment, correction, or smoothing technique(s) or method(s) in accordance with one or more aspects of the present disclosure;

FIG. 17 illustrates a block diagram of at least one embodiment of a continuum robot in accordance with one or more aspects of the present disclosure;

FIG. 18 illustrates a block diagram of at least one embodiment of a controller in accordance with one or more aspects of the present disclosure;

FIG. 19 shows a schematic diagram of an embodiment of a computer that may be used with one or more embodiments of an apparatus or system, or one or more methods, discussed herein in accordance with one or more aspects of the present disclosure; and

FIG. 20 shows a schematic diagram of another embodiment of a computer that may be used with one or more embodiments of an imaging apparatus or system, or methods, discussed herein in accordance with one or more aspects of the present disclosure.

### DETAILED DESCRIPTION OF THE PRESENT DISCLOSURE

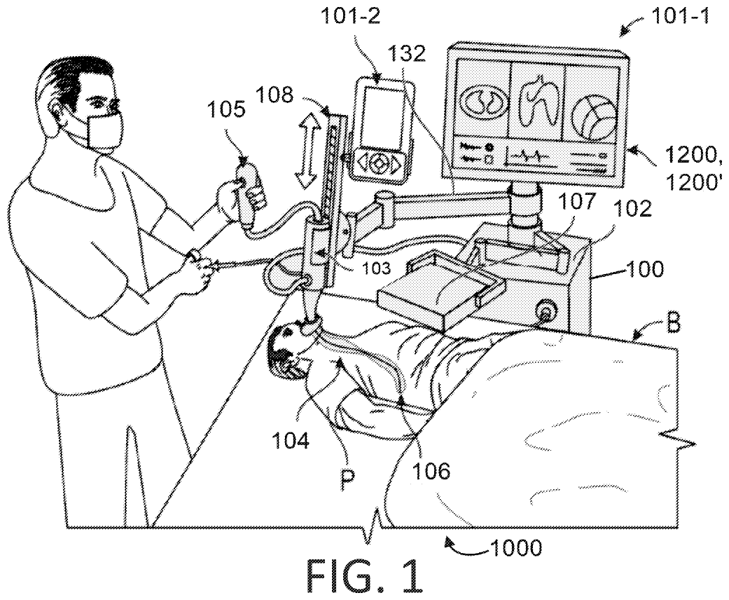

One or more devices, systems, methods and storage mediums for viewing, imaging, and/or characterizing tissue, or an object or sample, using one or more imaging techniques or modalities (such as, but not limited to, computed tomography (CT), Magnetic Resonance Imaging (MRI), any other techniques or modalities used in imaging (e.g., Optical Coherence Tomography (OCT), Near infrared fluorescence (NIRF), Near infrared auto-fluorescence (NIRAF), Spectrally Encoded Endoscopes (SEE)), etc.) are disclosed herein. Several embodiments of the present disclosure, which may be carried out by the one or more embodiments of an apparatus, system, method, and/or computer-readable storage medium of the present disclosure, are described diagrammatically and visually in FIGS. 1 through 20.

One or more embodiments of the present disclosure avoid the aforementioned issues by providing a simple and fast method or methods that provide adjustment, correction, and/or smoothing technique(s) as discussed herein.

Accordingly, it is a broad object of the present disclosure to provide imaging (e.g., computed tomography (CT), Magnetic Resonance Imaging (MRI), etc.) apparatuses, systems, methods, and storage mediums for using a navigation and/or control method or methods (manual or automatic) in one or more apparatuses or systems (e.g., an imaging apparatus or system, an endoscopic imaging device or system, a continuum robot, etc.). The navigation and/or control may be employed so that an apparatus or system having multiple portions or sections (e.g., multiple bending portions or sections) operates to: (i) keep track of a path of a portion (e.g., a tip) or of each of the multiple portions or sections of an apparatus or system; (ii) have a navigation and/or orientation (or pose) of each of the multiple portions or sections match a navigation and/or orientation (or pose) of a first portion or section of the multiple portions or sections in a case where each portion or section reaches or approaches a same or similar, or approximately same or similar, state, states, or position (e.g., a position or state(s) in a target, object, or specimen; a position or state(s) in a patient; a target position or state(s) in an image or frame; a set or predetermined position or state(s) in an image or frame; a set or predetermined position or state(s) in an image or frame where the first portion or section reaches or approaches the set or predetermined position or state(s) (or a position or state(s) that is approximately the same or similar to the set or predetermined position or state(s)) at one point in time and one or more of other portions or sections of the multiple portions or sections reach or approach the set or predetermined position or state(s) (or a position or state(s) that is approximately the same or similar to the set or predetermined position or state(s)) at one or more other points in time; any other state (which may include, but is not limited to, an orientation, a position, a pose, a navigation, a path (whether continuous or discontinuous), a state transition, any other desired motion(s) or combination of motion(s) (e.g., one or more features of the present disclosure may assist navigation, orientation, or any other types of motions discussed herein or as desired by a user), etc.), a combination of any state(s) and/or motion(s) discussed herein or desired by a user, etc.) of another portion or section of the one or more devices, systems, methods, and/or storage mediums of the present disclosure, etc.); and (iii) to address any discontinuous path that may occur (e.g., due to a change or movement of any portion or state(s) of the apparatus or system), for example, by smoothing out any difference in the discontinuous path over one or more multiple stage positions and/or state(s) (or position(s) and/or state(s) of another structure used for mapping path or path-like information).

One or more embodiments of the present disclosure avoid the aforementioned issues by: (i) providing a simple and fast method or methods that normalizes displaying or viewing images, and/or controlling navigation and/or orientation (e.g., rotational orientation, pose, etc.), of multiple portions or sections (e.g., multiple bending portions or sections) of an apparatus or system (e.g., a catheter, a probe, a continuum robot or portion thereof, an endoscope, a camera, etc.) so that the multiple portions or sections operate to have a navigation and/or orientation (or pose, or position, etc.) match a navigation and/or orientation (or pose, or position, etc.) in a case (or at a time or times) where each portion or section of the multiple portions or sections reaches or approaches a same or similar, or approximately same or similar, position or state(s) (e.g., a set or predetermined position or state(s), a target position or state(s), a position or state(s) being imaged, any other state or states (which may include, but is/are not limited to, an orientation, a position, a pose, a navigation, a path (whether continuous or discontinuous), a state transition, any other desired motion(s) or combination of motion(s) (e.g., one or more features of the present disclosure may assist navigation, orientation, or any other types of motions discussed herein or as desired by a user), etc.), a combination of any state(s) and/or motion(s) discussed herein or desired by a user, etc.) of another portion or section of the one or more devices, systems, methods, and/or storage mediums of the present disclosure, etc.); (ii) keeping track of a path of a portion of the apparatus or system to implement robotic control of a catheter or probe (e.g., by mapping the path or the apparatus or system to a stage position (or to other path-like information, such as, but not limited to, a position sensor in the catheter or probe (e.g., an EM sensor, another type of sensor, etc.)); and/or (iii) having any difference or discrepancy of a discontinuous path smoothed out over one or more multiple stage positions or over other path-like information (e.g., from a sensor, from an EM sensor, etc.). As such, physicians or other users of the apparatus or system may have reduced or saved labor and/or mental burden using the apparatus or system due to the state(s), such as, but not limited to, navigation, control, and/or orientation (or pose, or position, etc.) or other state(s) discussed herein (which may include, but is not limited to, an orientation, a position, a pose, a navigation, a path (whether continuous or discontinuous), a state transition, any other desired motion(s) or combination of motion(s) (e.g., one or more features of the present disclosure may assist navigation, orientation, or any other types of motions discussed herein or as desired by a user), a combination of any state(s) and/or motion(s) discussed herein or desired by a user, etc.), feature(s) of the present disclosure. In one or more embodiments of the present disclosure, a labor of a user to control and/or navigate (e.g., rotate, translate, etc.) the imaging apparatus or system or a portion thereof (e.g., a catheter, a probe, a continuum robot, a camera, one or more sections or portions of a catheter, probe, continuum robot, camera, etc.) is saved or reduced.

In one or more embodiments of an imaging device or system, or a portion of the imaging device or system (e.g., a catheter, a probe, continuum robot, etc.), the multiple sections or portions may be multiple bending sections or portions. In one or more embodiments, the imaging device or system may include manual and/or automatic navigation and/or control features. For example, a user of the imaging device or system may control each section or portion, and/or the imaging device or system may operate to automatically control (e.g., robotically control) each section or portion. In one or more embodiments, a device or system may provide a combination of automatic and manual control where the device or system may automatically control a portion or portions of a continuum robot, and the device or system may allow a user of the device or system to make manual adjustments to the control (e.g., to a path) of the continuum robot.

Navigation, control, and/or orientation feature(s) may include, but are not limited to, implementing mapping of a pose (angle value(s), plane value(s), etc.) of a first portion or section (e.g., a tip portion or section, a distal portion or section, a predetermined or set portion or section, a user selected or defined portion or section, etc.) to a stage position/state (or a position/state of another structure used for mapping path or path-like information), controlling angular position(s) or state(s) of one or more of the multiple portions or sections, controlling rotational orientation(s), position(s), or state(s) of one or more of the multiple portions or sections, controlling (manually or automatically (e.g., robotically)) one or more other portions or sections of the imaging device or system to match or substantially or approximately match (or be close or similar to) the navigation/orientation/position/pose/state of the first portion or section in a case where the one or more other portions or sections reach (e.g., subsequently reach, reach at a different time, etc.) the same or similar, or approximately the same or similar, position or state (e.g., in a target, in an object, in a sample, in a patient, in a frame or image, etc.) during navigation in or along a first direction of a path of the imaging device or system, controlling each of the sections or portions of the imaging device or system to retrace and match (or substantially or approximately match or be close/similar to) prior respective position(s) of the sections or portions in a case where the imaging device or system is moving or navigated in a second direction (e.g., in an opposite direction along the path, in a return direction along the path, in a retraction direction along the path, etc.) along the path, etc. For example, an imaging device or system (or portion thereof, such as, but not limited to, a probe, a catheter, a camera, etc.) may enter a target along a path where a first section or portion of the imaging device or system (or portion of the device or system) is used to set the navigation, control, or state path and state(s)/position(s), and each subsequent section or portion of the imaging device or system (or portion of the device or system) is controlled to follow the first section or portion such that each subsequent section or portion matches (or is similar to, approximate to, substantially matching, etc.) the orientation, position, state, etc. of the first section or portion at each location along the path. During retraction, each section or portion of the imaging device or system is controlled to match (or be similar to, be approximate to, be substantially matching, etc.) the prior orientation, position, state, etc. (for each section or portion) for each of the locations along the path. In other words, each section or portion of the device or system may follow a leader (or more than one leader) or may use one or more FTL technique(s) discussed herein. As such, an imaging or continuum robot device or system (or catheter, probe, camera, etc. of the device or system) may enter and exit a target, an object, a specimen, a patient (e.g., a lung of a patient, an esophagus of a patient, another portion of a patient, another organ of a patient, a vessel of a patient, etc.), etc. along the same, similar, approximately same or similar, etc. path and using the same orientation, position, state, etc. for entrance and exit to achieve an optimal navigation, orientation, control, and/or state path. The navigation, control, orientation, and/or state feature(s) are not limited thereto, and one or more devices or systems of the present disclosure may include any other desired navigation, control, orientation, and/or state specifications or details as desired for a given application or use. In one or more embodiments and while not limited thereto, the first portion or section may be a distal or tip portion or section of the imaging or continuum robot device or system. In one or more embodiments, the first portion or section may be any predetermined or set portion or section of the imaging or continuum robot device or system and may be predetermined or set manually by a user of the imaging or continuum robot device or system and/or may be set automatically by the imaging device or system (or by a combination of manual and automatic control).

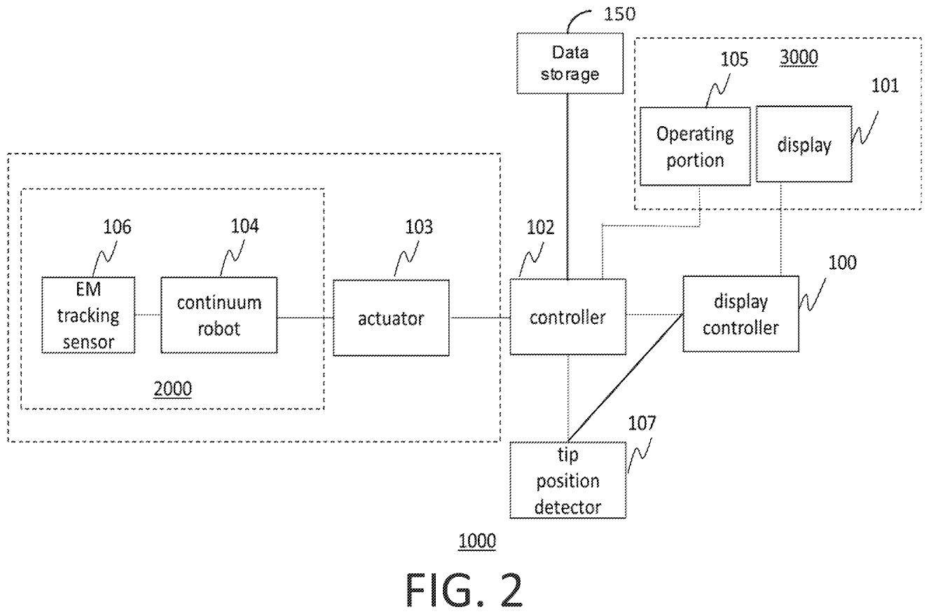

As shown in FIGS. 1-4 of the present disclosure, one or more embodiments of a system 1000 for performing image adjustment, correction, and/or smoothing (e.g., for a continuum robot) may include one or more of the following: a display controller 100, a display 101-1, a display 101-2, a controller 102, an actuator 103, a continuum device 104, an operating portion 105, an EM tracking sensor 106, a catheter tip position/orientation/pose/state detector 107, and a rail 108 (for example, as shown in at least FIGS. 1-2). The system 1000 may include one or more processors, such as, but not limited to, a display controller 100, a controller 102, a CPU 120, a controller 50, a CPU 51, a console or computer 1200 or 1200′, a CPU 1201, any other processor or processors discussed herein, etc., that operate to execute a software program, to control the one or more adjustment, control, and/or smoothing technique(s) discussed herein, and to control display of a navigation screen on one or more displays 101. The one or more processors (e.g., the display controller 100, the controller 102, the CPU 120, the controller 50, the CPU 51, the console or computer 1200 or 1200′, the CPU 1201, any other processor or processors discussed herein, etc.) may generate a three dimensional (3D) model of a structure (for example, a branching structure like airway of lungs of a patient, an object to be imaged, tissue to be imaged, etc.) based on images, such as, but not limited to, CT images, MRI images, etc. Alternatively, the 3D model may be received by the one or more processors (e.g., the display controller 100, the controller 102, the CPU 120, the controller 50, the CPU 51, the console or computer 1200 or 1200′, the CPU 1201, any other processor or processors discussed herein, etc.) from another device. A two-dimensional (2D) model may be used instead of 3D model in one or more embodiments. The 2D or 3D model may be generated before a navigation starts. Alternatively, the 2D or 3D model may be generated in real-time (in parallel with the navigation). In the one or more embodiments discussed herein, examples of generating a model of branching structure are explained. However, the models may not be limited to a model of branching structure. For example, a model of a route direct to a target may be used instead of the branching structure. Alternatively, a model of a broad space may be used, and the model may be a model of a place or a space where an observation or a work is performed by using a continuum robot 104 explained below.

While not limited to such a configuration, the display controller 100 may acquire position/orientation/navigation/pose/state (or other state) information of the continuum robot 104 from a controller 102. Alternatively, the display controller 100 may acquire the position/orientation/navigation/pose/state (or other state) information directly from a tip position/orientation/navigation/pose/state (or other state) detector 107. The continuum robot 104 may be a catheter device (e.g., a steerable catheter or probe device). The continuum robot 104 may be attachable/detachable to the actuator 103, and the continuum robot 104 may be disposable.

In one or more embodiments, the one or more processors, such as, but not limited to, the display controller 100, may generate and output a navigation screen to the one or more displays 101-1, 101-2 based on the 2D/3D model and the position/orientation/navigation/pose/state (or other state) information by executing the software. The navigation screen indicates a current position/orientation/navigation/pose/state (or other state) of the continuum robot 104 on the 2D/3D model. By using the navigation screen, a user may recognize the current position/orientation/navigation/pose/state (or other state) of the continuum robot 104 in the branching structure.

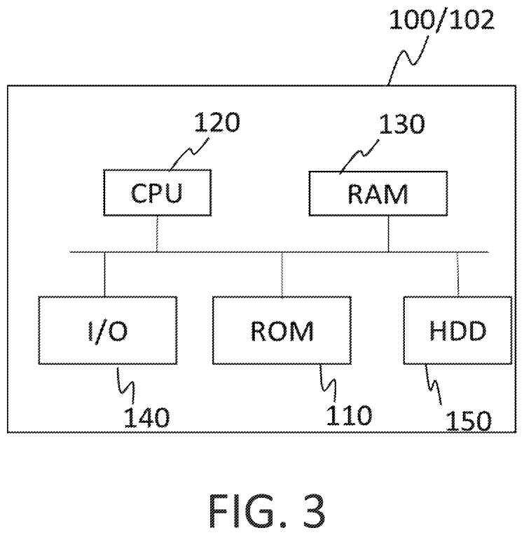

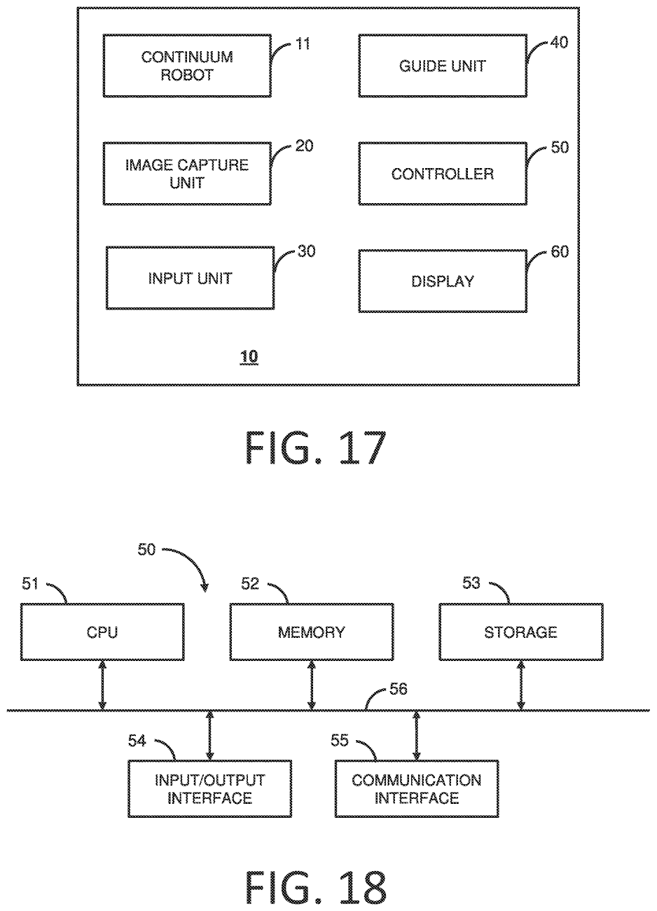

In one or more embodiments, the one or more processors, such as, but not limited to, the display controller 100 and/or the controller 102, may include, as shown in FIG. 3, at least one storage Read Only Memory (ROM) 110, at least one central processing unit (CPU) 120, at least one Random Access Memory (RAM) 130, at least one input and output (I/O) interface 140 and at least one Hard Disc Drive (HDD) 150 (see e.g., also data storage 150 of FIG. 2). A Solid State Drive (SSD) may be used instead of HDD 150 as the data storage 150. In one or more additional embodiments, the one or more processors, and/or the display controller 100 and/or the controller 102, may include structure as shown in FIGS. 17-18 and 19-20 as further discussed below.

The ROM 110 and/or HDD 150 operate to store the software in one or more embodiments. The RAM 130 may be used as a work memory. The CPU 120 may execute the software program developed in the RAM 130. The I/O 140 operates to input the positional (or other state) information to the display controller 100 (and/or any other processor discussed herein) and to output information for displaying the navigation screen to the one or more displays 101-1, 101-2. In the embodiments below, the navigation screen may be generated by the software program. In one or more other embodiments, the navigation screen may be generated by a firmware.

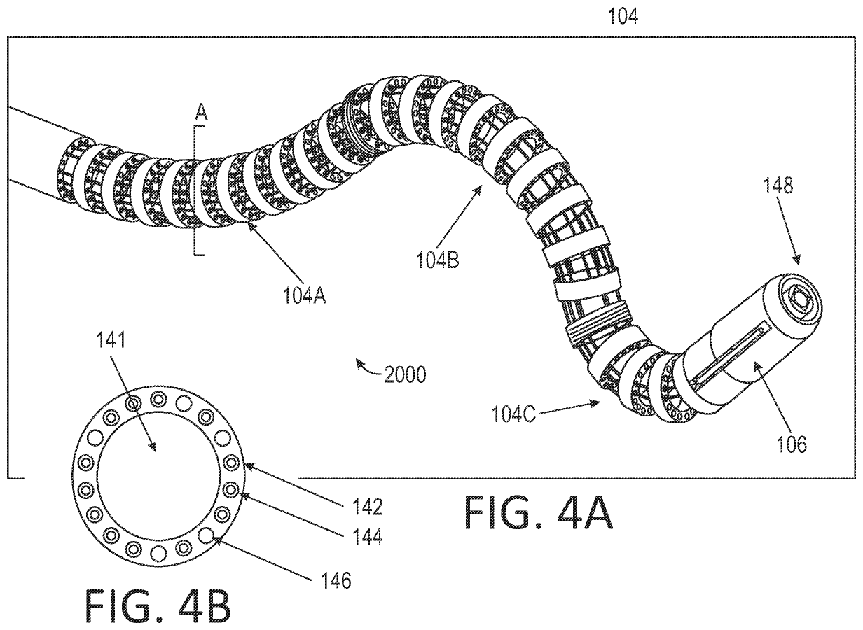

FIGS. 4A-4B show at least one embodiment of a continuum robot 104 that may be used in the system 1000 or any other system discussed herein. The continuum robot 104 may include a proximal section, a middle section, and a distal section, and each of the sections may be bent by a plurality of drive or driving wires (driving liner members, such as a driving backbone or backbones). In one or more embodiments, the continuum robot may be a catheter device 104. The posture of the catheter device 104 may be supported by supporting wires (supporting liner members, for example, passive sliding backbones). The drive or driving wires may be connected to the actuator 103. The actuator 103 may include one or more motors and drives for each of the sections of the catheter 104 by pushing and/or pulling the drive or driving wires (driving backbones). The actuator 103 may proceed or retreat along a rail 108 (e.g., to translate the actuator 103, the continuum robot/catheter 104, etc.) moved by a motorized linear stage, and the actuator 103 and continuum robot 104 may proceed or retreat in and out of the patient's body or other target, object, or specimen (e.g., tissue). As shown in FIG. 4B, the catheter device 104 may include a plurality of driving backbones and may include a plurality of passive sliding backbones. In one or more embodiments, the catheter device 104 may include at least nine (9) driving backbones and at least six (6) passive sliding backbones. The catheter device 104 may include an atraumatic tip at the end of the distal section of the catheter device 104.

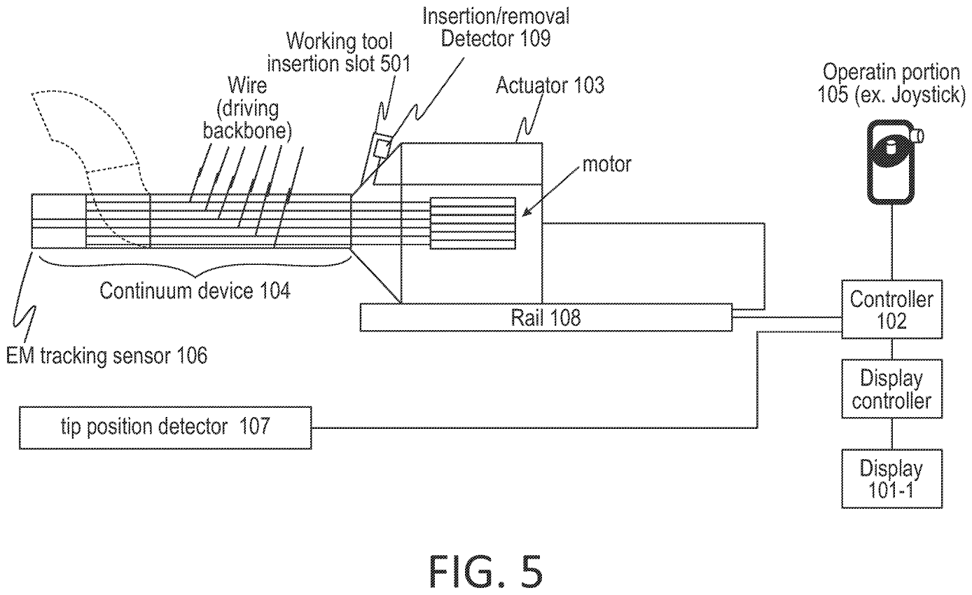

In some embodiments, the continuum robot 104 may comprise a distal bending section 104C, a middle bending section 104B, and a proximal bending section 104A. Embodiments with further bending sections (controlled by additional driving wires) as well as an embodiment with only two bending sections are also contemplated in the present disclosure. The control of the bending sections of the continuum robot 104 may be achieved by the use of driving wires. These driving wires (see, for example, 142 and 144 in FIG. 4B) may run from the distal portion of a bending section (e.g., section(s) 104A, 104B, 104C, etc.) to an actuator and/or motor (see e.g., as shown in the embodiment of FIG. 5) to control the orientation/pose/position/state/any combination thereof/etc. of the bending section (e.g., section(s) 104A, 104B, 104C, etc.). As shown in FIG. 4A, and while not limited to the shown number of drive wires (also referred to herein as driving wires), the distal bending section 104C may have three drive wires to control the orientation/pose/position/state/any combination thereof/etc. of the bending section 104C. Similarly, the middle bending section 104B may have three drive wires to control the orientation/pose/position/state/any combination thereof/etc. of the middle bending section 104B, where the guide ring(s) may also have the drive wires from the distal bending section running through this section of the continuum robot 104. The proximal bending section 104A may have three additional drive wires controlling the orientation/pose/position/state/any combination thereof/etc. of the proximal bending section 104A.

One or more embodiments of the catheter/continuum robot 104 may include an electro-magnetic (EM) tracking sensor 106. One or more other embodiments of the catheter/continuum robot 104 may not include or use the EM tracking sensor 106. The electro-magnetic tracking sensor (EM tracking sensor) 106 may be attached to the tip of the continuum robot 104. In this embodiment (as shown in FIGS. 4A-4B), a robot 2000 may include the continuum robot 104 and the EM tracking sensor 106 (as also seen diagrammatically in FIG. 2), and the robot 2000 may be connected to the actuator 103.

One or more devices or systems, such as the system 1000, may include a tip position/orientation/navigation/pose/state (or other state) detector 107 that operates to detect a position/orientation/navigation/pose/state (or other state) of the EM tracking sensor 106 and to output the detected positional (and/or other state) information to the controller 100 or 102 (e.g., as shown in FIGS. 2-3), or to any other processor(s) discussed herein.

The controller 102 operates to receive the positional (or other state) information of the tip of the continuum robot 104 from the tip position/orientation/navigation/pose/state (or any other state discussed herein) detector 107. The controller 100 and/or the controller 102 operates to control the actuator 103 in accordance with the manipulation by a user (e.g., manually), and/or automatically (e.g., by a method or methods run by one or more processors using software, by the one or more processors, using automatic manipulation in combination with one or more manual manipulations or adjustments, etc.) via one or more operation/operating portions or operational controllers 105 (e.g., such as, but not limited to a joystick as shown in FIG. 1 or FIG. 5; see also, diagram of FIG. 2). The one or more displays 101-1, 101-2 and/or operation portion or operational controllers 105 may be used as a user interface 3000 (also referred to as a receiving device) (e.g., as shown diagrammatically in FIG. 2). In an embodiment shown in FIGS. 1-2 or the embodiment shown in FIG. 5, the system(s) 1000 may include, as an operation unit, the display 101-1 (e.g., such as, but not limited to, a large screen user interface with a touch panel, first user interface unit, etc.), the display 101-2 (e.g., such as, but not limited to, a compact user interface with a touch panel, a second user interface unit, etc.) and the operating portion 105 (e.g., such as, but not limited to, a joystick shaped user interface unit having shift lever/button, a third user interface unit, a gamepad, or other input device, etc.).

The controller 100 and/or the controller 102 (and/or any other processor discussed herein) may control the continuum robot 104 based on an algorithm known as follow the leader (FTL) algorithm. By applying the FTL algorithm, the middle section and the proximal section (following sections) of the continuum robot 104 may move at a first position (or other state) in the same or similar way as the distal section moved at the first position (or other state) or a second position (or state) near the first position (or state) (e.g., during insertion of the continuum robot/catheter 104). Similarly, the middle section and the distal section of the continuum robot 104 may move at a first position or state in the same/similar/approximately similar way as the proximal section moved at the first position or state or a second position or state near the first position (e.g., during removal of the continuum robot/catheter 104). Alternatively, the continuum robot/catheter 104 may be removed by automatically and/or manually moving along the same or similar, or approximately same or similar, path that the continuum robot/catheter 104 used to enter a target (e.g., a body of a patient, an object, a specimen (e.g., tissue), etc.) using the FTL algorithm, including, but not limited to, using FTL with the one or more adjustment, correction, state, and/or smoothing technique(s) discussed herein.

During navigation of the continuum robot 104 with the FTL algorithm in one or more embodiments, an operator or user may control the distal section for an orientation of the continuum robot 104 and the rail 108 for an insertion depth by using operation portion 105. The operator control or process may comprise or include the following two steps:
(1) An insertion step: in one or more embodiments, the operator or user may move the rail 108 to insert the continuum robot 104 (e.g., into an object, target, or sample; into the patient P; into a vessel of the patient P; into an airway(s) or lung(s) of the patient P; into another portion of the patient P; etc.). During the insertion motion, the FTL algorithm may control the middle and proximal sections and make the middle and proximal sections follow a leader section (e.g., a tip, the distal section, a tip of the distal section, another leader section discussed herein, etc.).
(2) An exploration step: in one or more embodiments, the operator or user may move the distal section, a tip, a tip of the distal section, any other leader section discussed herein, etc. to determine a final orientation for the operator or user to insert the continuum robot 104 (e.g., into an object, target, or sample; into the patient P; etc.) at or along one position of the rail 108.

In a typical workflow to navigate the continuum robot 104 (e.g., into an object, target, or sample; into the patient P; into a vessel of the patient P; into an airway(s) or lung(s) of the patient P; into another portion of the patient P; etc.), the operator or user may insert the continuum robot 104 with the insertion step until a next bifurcation of the object, target, or sample (e.g., an object, target, or sample discussed herein; the patient P; a vessel of the patient P; an airway(s) or lung(s) of the patient P; another portion of the patient P; etc.). Then, the operator or user may stop the rail 108 and may migrate or proceed to the exploration step. In one or more embodiments, the insertion step and the exploration step may be performed or may occur simultaneously. In the exploration step, the operator or user may move the leader/distal section (or any other leader section or portion of a leader or distal section (e.g., a tip thereof) discussed herein) with the endoscopic view, explore the orientation that may be used to insert the continuum robot 104, and determine the orientation of the leader/distal section to the target airway (in a case where the continuum robot 104 is inserted int an airway or lung of the patient P) or to any other predetermined or desired object, target, or sample (e.g., an object, target, or sample discussed herein; the patient P; a vessel of the patient P; an airway(s) or lung(s) of the patient P; another portion of the patient P; etc.). Then, the operator or user may start or repeat the insertion step again to reach any following bifurcation.

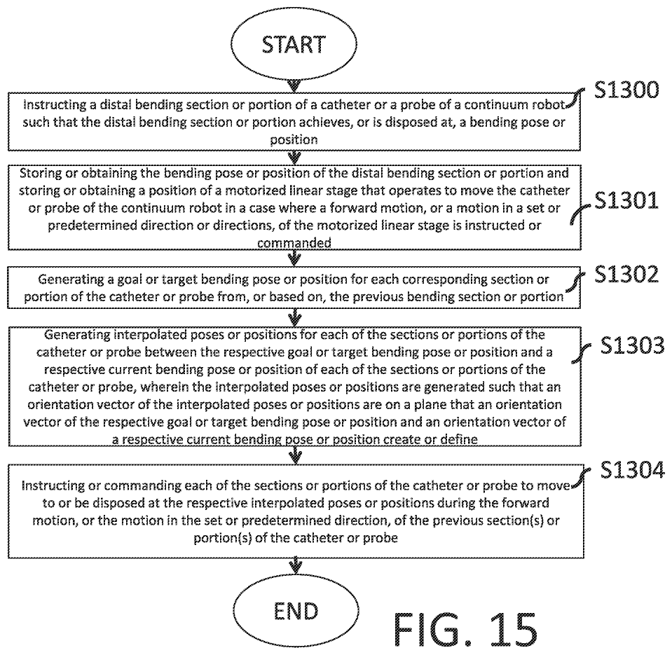

During this workflow, in one or more embodiments, the system or apparatus, one or more processors thereof, etc. may perform the flowchart in FIG. 15. A start state in FIG. 15 may be the beginning of the exploration step in at least one embodiment of the workflow. The system receives an instruction of the distal section (or the leader section, in one or more embodiments) bending and executes the bending (see e.g., step S1300 in FIG. 15). Then, the operator or user may start the insertion step after determining the distal (or leader) section orientation/pose/state/any combination thereof/etc. At this time, the system, one or more processors thereof, etc. may store the bending orientation/pose/state/any combination thereof/etc. of the distal (or leader) section while starting to move the rail 108 as per the operator's or user's instruction (see e.g., step S1301 in FIG. 15). In one or more embodiments, the system or apparatus, one or more processors thereof, etc. may update a lookup table in a case where, or every time, the distal section or portion bends (regardless of whether or not the rail 108 or a linear stage is moving). After S1301, the system may generate a goal (target) bending orientation/pose/state/any combination thereof/etc. for each middle and proximal section (see e.g., step S1302 in FIG. 15). This goal bending orientation/pose/state/any combination thereof/etc. may be used for the FTL algorithm and may be the same orientation/pose/state/any combination thereof/etc. as the leader section for each middle and proximal section after the insertion by the section length of each middle and proximal section. For example, the middle section may achieve the same orientation/pose/state/any combination thereof/etc. as the distal (or leader) section in a case where the middle section reaches the previous insertion position of the distal (or leader) section during the insertion with the FTL algorithm.

Then, the system or apparatus, the one or more processors thereof, etc. may generate interpolated orientations, poses, states, any combination(s) thereof, etc. for each middle and proximal section between the current and goal bending orientations, poses, states, any combination(s) thereof, etc. (see e.g., step S1303 of FIG. 15). Since the system, apparatus, etc. embodiments of the present disclosure may have an internal minimal insertion interval as an insertion control resolution, the system, apparatus, etc. embodiments of the present disclosure may generate these interpolated orientations, poses, states, any combination(s) thereof, etc. by the minimal insertion interval. Then, with step S1304 of FIG. 15, the system, apparatus, one or more processors thereof, etc. embodiments of the present disclosure may control all sections smoothly along the FTL algorithm through the insertion by the minimal insertion length.

In one or more embodiments, the system, apparatus, one or more processors thereof, etc. may express the bending orientation/pose/state/any combination thereof/etc. by using drive or driving wire positions. In one or more embodiments, the current and goal bending orientations, poses, states, any combination(s) thereof, etc. may be converted to the corresponding driving wire positions for each section. Then, the system, apparatus, one or more processors thereof, etc. may interpolate the driving wire positions between the current and goal driving wire positions. As explained with FIGS. 4A and 4B, each section may have three driving wires terminated at the distal end of each section. As shown in FIG. 4B, each section may have three driving wires at the fixed positions of the sub-lumens along a circumference of the guide ring. In step S1301, the system, apparatus, one or more processors thereof, etc. may store the three driving wire positions of the distal section. Then, in step S1302, the system, apparatus, one or more processors thereof, etc. may compute the goal driving wire positions for a middle section or portion (or a proximal section or portion) out of, or based on, the recorded driving wire positions of the distal section. In one or more embodiments, the system or apparatus, one or more processors thereof, etc. may: (i) convert current and goal bending poses, positions, or states to corresponding drive or driving wire positions for each section or portion of the catheter or probe, (ii) determine the drive or driving wire positions of the distal bending section or portion, the middle bending section or portion, and/or the proximal bending section or portion, and (iii) (a) compute the goal drive or driving wire positions for at least a middle section or portion and a proximal section or portion of the catheter or probe out of, or based on, the drive or driving wire positions of the distal bending section or portion, or (b) select one of the distal bending section or portion, the middle bending section or portion, and the proximal bending section or portion and compute the goal drive or driving wire positions for the other two sections or portions of the catheter or probe out of, or based on, the selected one of the distal bending section or portion, the middle bending section or portion, and/or the proximal bending section or portion (in other words, any section or portion of the catheter or probe may be selected and used to compute the goal drive or driving wire positions for any of the other sections or portions of the catheter or probe such that the distal, middle, and/or proximal sections or portions (or any other section or portion) of the catheter or probe may be swapped or interchangeable for such computations/determinations). By way of a further example, the orientation of the distal section/position of a distal wire may be calculated from the orientation of the middle section/position of a middle wire (and/or may be calculated from the orientation of the proximal section/position of a proximal wire). For this computation, the system, apparatus, one or more processors thereof, etc. may use the cross-sectional position difference of the three driving wires between the distal and middle sections since the positions of the three driving wires for a bending orientation, pose, state, any combination thereof, etc. may be covered or converted into the positions of another three driving wires at and/or using the difference cross-sectional position. In step S1303, the system, apparatus, one or more processors thereof, etc. may create linear interpolation of the driving wire positions between the current and goal driving wire positions of the middle section for every minimal insertion interval. In one or more embodiments, a method may take the difference between the current and goal drive or driving wire positions mapped to the target stage position, and a linear interpolation of the difference may be created and added to the corresponding values already stored at every minimal insertion level.

By generating the interpolated driving wire positions for insertion with the FTL algorithm, each section may bend at a constant rate through the insertion since a change of the driving wire position from one minimal insertion interval to another adjacent minimal insertion interval may be constant in one or more embodiments or may always be constant (if desired in a particular system embodiment, apparatus embodiment, etc.). The constant rate of each section's orientation/pose/state/etc. change may give the operator or user a constant change of the endoscopic view for intuitive control. In one or more embodiments, the same or constant rate of change, or a substantially same/similar or constant rate of change, of the driving wire position may correspond to a same or constant (or to a substantially same/similar or constant) speed or velocity for the distal/leader section or portion (or tip of the distal/leader section or portion) of the catheter or probe 104. Also, since each section may approach from the current orientation/pose/position/state/etc. to the goal orientation/pose/position/state/etc. with the constant rate, the trajectory of the distal (or leader) section through the insertion may route along the shorter path. With this feature, one or more embodiments of the continuum robot 104 may reduce or avoid an interaction to or with any predetermined portion of the target, object, or sample (e.g., in a case of a lung or airway, interaction with or to the airway walls may be reduced or avoided). Additionally, in a case where the difference between the current and goal drive or driving wire positions mapped to the target stage position is taken and the linear interpolation of the difference may be created and added to the corresponding values already stored at every minimal insertion level, an additional feature may be achieved where the additional feature includes adding a portion of the total wire position different to each point along the interpolated range so that the target pose, position, state, etc. gradually aims towards the final target as the pose, position, state, etc. approaches the target, while also maintaining any previously mapped poses, positions, states, etc. The subject feature may ensure the section or portion will still follow any necessary, desired, or target bends during the approach to the target position and still operates to minimize interaction with an object, target, or sample (such as, but not limited to, minimizing interaction with an airway or with airway wall(s) at those locations in a case where the object, target, or sample is an airway, a lung, etc.).

Any of the one or more processors, such as, but not limited to, the controller 102 and the display controller 100, may be configured separately. As aforementioned, the controller 102 may similarly include a CPU 120, a RAM 130, an I/O 140, a ROM 110, and a HDD 150 as shown diagrammatically in FIG. 3. Alternatively, any of the one or more processors, such as, but not limited to, the controller 102 and the display controller 100, may be configured as one device (for example, the structural attributes of the controller 100 and the controller 102 may be combined into one controller or processor, such as, but not limited to, the one or more other processors discussed herein (e.g., computer, console, or processor 1200, 1200′, etc.).

The system 1000 may include a tool channel for a camera, biopsy tools, or other types of medical tools (as shown in FIG. 1 or FIG. 5). For example, the tool may be a medical tool, such as an endoscope, a forceps, a needle or other biopsy tools, etc. In one or more embodiments, the tool may be described as an operation tool or working tool. The working tool may be inserted or removed through a working tool insertion slot 501 (as shown in FIG. 5). Any of the features of the present disclosure may be used in combination with any of the features, including, but not limited to, the tool insertion slot 501, as discussed in U.S. Prov. Pat. App. No. 63/378,017, filed Sep. 30, 2022, the disclosure of which is incorporated by reference herein in its entirety, and/or any of the features as discussed in U.S. Prov. Pat. App. No. 63/377,983, filed Sep. 30, 2022, the disclosure of which is incorporated by reference herein in its entirety.

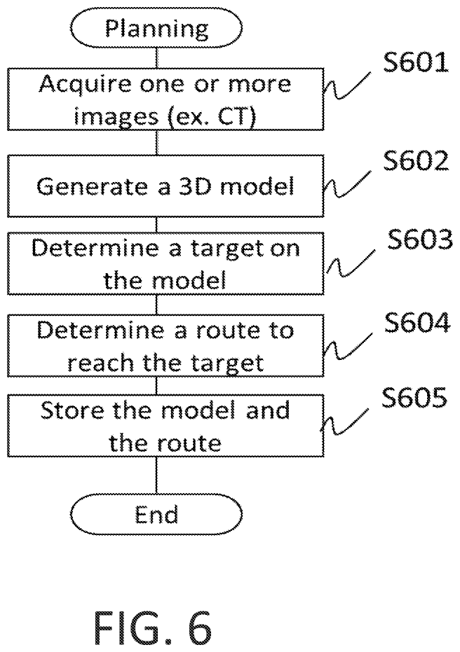

One or more of the features discussed herein may be used for planning procedures. As an example of one or more embodiments, FIG. 6 is a flowchart showing steps of at least one planning procedure of an operation of the continuum robot/catheter device 104. One or more of the processors discussed herein may execute the steps shown in FIG. 6, and these steps may be performed by executing a software program read from a storage medium, including, but not limited to, the ROM110 or HDD 150, by CPU 120 or by any other processor discussed herein. One or more methods of planning using the continuum robot/catheter device 104 may include one or more of the following steps: (i) In step s601, one or more images such, as CT or MRI images, may be acquired; (ii) In step s602, a three dimensional model of a branching structure (for example, an airway model of lungs or a model of an object, specimen or other portion of a body) may be generated based on the acquired one or more images; (iii) In step s603, a target on the branching structure may be determined (e.g., based on a user instruction, based on preset or stored information, etc.); (iv) In step s604, a route of the continuum robot/catheter device 104 to reach the target (e.g., on the branching structure) may be determined (e.g., based on a user instruction, based on preset or stored information, based on a combination of user instruction and stored or preset information, etc.); (v) In step s605, the generated model (e.g., the generated two-dimensional or three-dimensional model) and the decided route on the model may be stored (e.g., in the RAM 130 or HDD or data storage 150, in any other storage medium discussed herein, in any other storage medium known to those skilled in the art, etc.). In this way, a model (e.g., a 2D or 3D model) of a branching structure may be generated, and a target and a route on the model may be determined and stored before the operation of the continuum robot 104 is started.

In one or more of the embodiments below, embodiments of using a catheter device/continuum robot 104 are explained, such as, but not limited to features for performing adjustment, correction, and/or smoothing (e.g., direct FTL smoothing, continuum robot smoothing, path smoothing, or other smoothing discussed herein) technique(s).

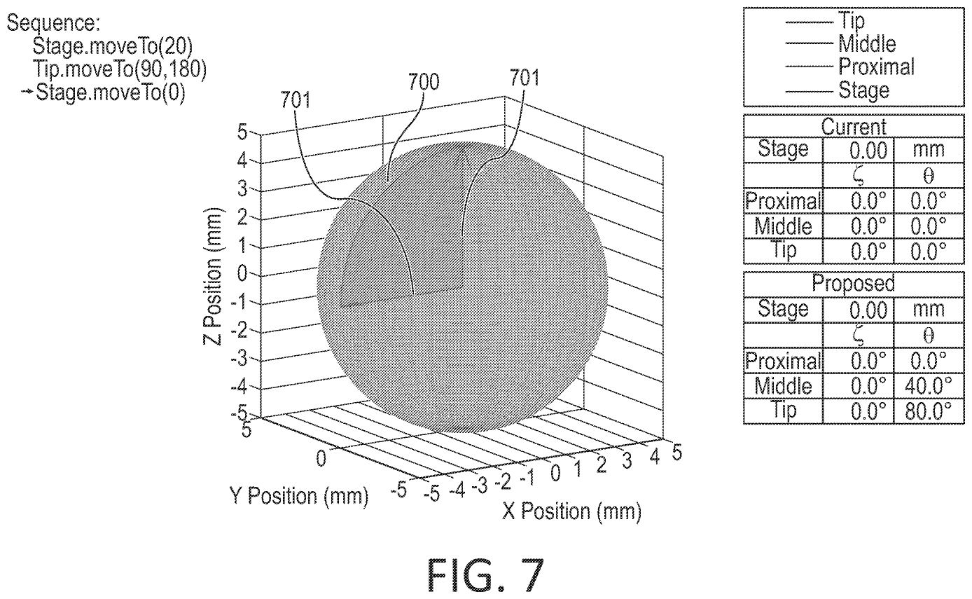

In one or more embodiments, an effective manner of smoothing may be to aim a section (e.g., a distal or tip portion or section) in a direct path between orientations. In one or more embodiments, the direct path may be the shortest path (see e.g., a path 700 as shown in FIGS. 7-9) and may use a smallest possible volume for efficiency. In one or more embodiments, a shortest path may reside on a plane defined by two vectors (e.g., a plane defined a start vector 701 an end vector 701 as shown in FIG. 7, a plane defined by a base coordinate system having the two vectors in the plane such that the shortest path 700 is resulting from or defined by the other two vectors 701, 701, etc.). The shortest path 700 is preferably employed for efficiency of use and control of the device or system of the continuum robot (e.g., the continuum robot 104).

In contrast, the path 700 is shorter than (or considered the shortest) another path 800 (as shown in FIG. 8) which may be used for one or more other continuum robot embodiments. As shown in FIG. 8, the path 800 is outside of the plane of the two vectors 701, is longer than the path 700, and would employ the indirect path 800, which may be used alternatively to path 700 or which may be adjusted to arrive at path 700 (e.g., by transforming or adjusting path 800 to match path 700) in one or more embodiments of the present disclosure. In one or more embodiments, an orientation or state may include one or more degrees of freedom. For example, in at least one orientation embodiment, two (2) degrees of freedom may be used, which may include an angle for a magnitude of bending and a plane for a direction of bending. In one or more embodiments, matching state(s) may involve matching, duplicating, mimicking or otherwise copying other characteristics, such as, but not limited to, vectors for each section or portion of the one or more sections or portions of a probe or catheter, for different portions or sections of the catheter or probe. For example, a transition or change from a base angle/plane to a target angle/plane may be set or predetermined using transition values (e.g., while not limited hereto, a base orientation or state may have a stage at 0 mm, an angle at 0 degrees, and a plane at 0 degrees whereas a target orientation or state may have a stage at 20 mm, an angle at 90 degrees, and a plane at 180 degrees. The intermediate values for the stage, angle, and plane may be set depending on how many transition orientations or states may be used). By way of at least one example, in a case where five transition orientations or states may be used, a base orientation or state may have a stage at 0 mm, an angle at 0 degrees, and a plane at 0 degrees, and the target orientation or state may have a stage at 20 mm, an angle at 90 degrees, and a plane at 180 degrees as aforementioned. The three middle or intermediate orientations or states may have respective values as follows: (i) a 25% orientation or state may have a stage at 5 mm, an angle at 22.5 degrees, and a plane at 45 degrees; (ii) a 50% orientation or state may have a stage at 10 mm, an angle at 45 degrees, and a plane at 90 degrees; and (iii) a 75% orientation or state may have a stage at 15 mm, an angle at 67.5 degrees, and a plane at 135 degrees. The one or more features of the present disclosure reduce or minimize out of plane motion and surface area for actions taken by a probe or catheter device or system in one or more embodiments.

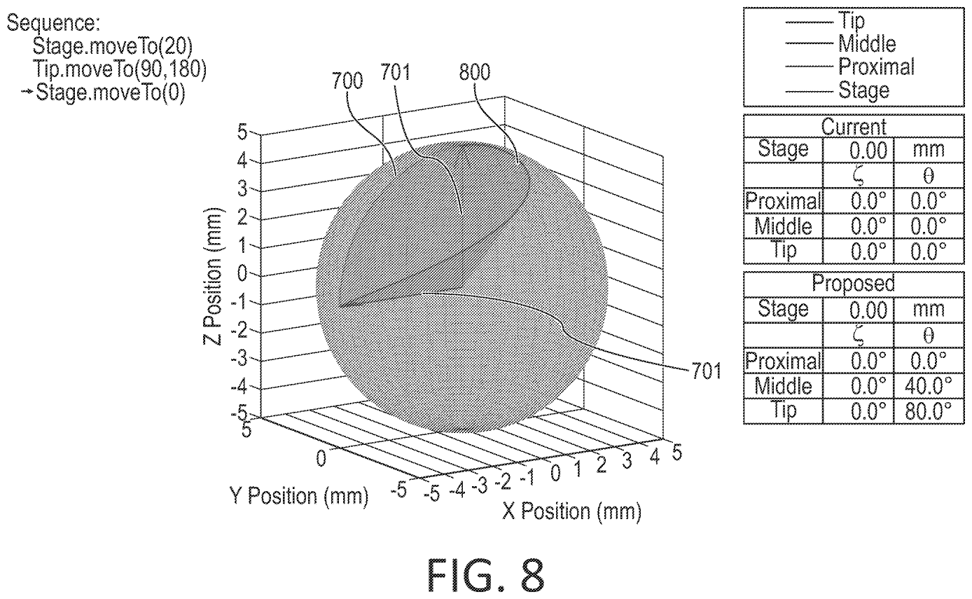

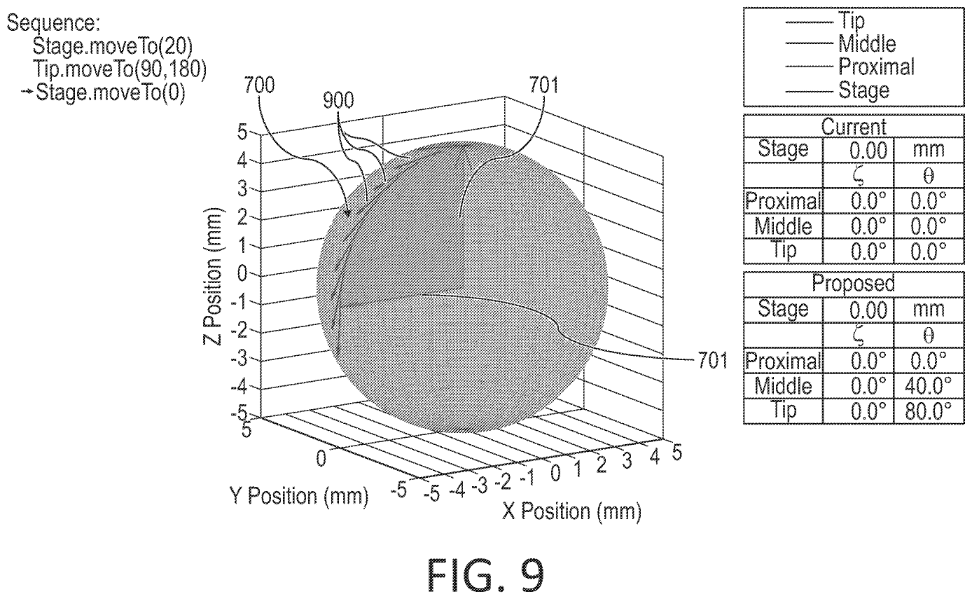

In one or more embodiments, motion along a path (e.g., the path 700) may be in a continuous direction as shown by the vectors 900 in FIG. 9.

One or more embodiments may use indirect path(s). In a case where an indirect path may be used, a motion (e.g., of one portion or section, of a first portion or section, etc.) may also affect subsequent portions or sections of the catheter or probe of a device or continuum robot (e.g., such as, but not limited to, the device/continuum robot 104). In a case where a direct path may be used, the motion and any resulting effect on subsequent portions or sections is reduced and/or minimized. Such a reduction or minimization is important and useful for a user of the catheter or probe of a device or continuum robot (e.g., such as, but not limited to, the device/continuum robot 104) or is useful for a continuum robot system or apparatus (such as, but not limited to, the continuum robot 104, the system or apparatus 1000 of FIGS. 1-2, the system or apparatus 100 discussed further below, the system or apparatus 100′ discussed further below, the system or apparatus 100″ discussed further below, etc.) that is automatically (in part (e.g., manual control feature(s) may be included or provided) or in whole) controlling the catheter or probe in a case where a camera is at the very end or Tip portion or section, or close to an end or Tip portion or section, of the catheter or probe. To reduce and/or avoid an undesired motion (e.g., that may be caused by an indirection motion that may affect the Tip section or other portion of the catheter or probe), a user may adjust the Tip portion or section orientation (e.g., manually), or the system or apparatus may operate (e.g., via one or more processors thereof) to adjust the Tip portion or section orientation automatically. In one or more embodiments, in a case where an indirect path approaches a final orientation, the orientation of the catheter or probe moving along the indirect path may use an adjustment (e.g., the adjustment to the Tip portion or section orientation, an adjustment to another predetermined or set portion or section orientation, etc.). An additional adjustment may be used in a case where the orientation adjustment (e.g., the adjustment to the Tip portion or section orientation, the adjustment to another predetermined or set portion or section orientation, etc.) may be undone or removed by the additional adjustment (e.g., in a case where the original or initial orientation adjustment may have been avoided or may have been unnecessary, etc.). Since the orientations may be stored in history (e.g., in a memory or other storage medium such as, but not limited to, the memories or storage mediums discussed herein, memories known to those skilled in the art, etc.), the orientations may be repeated by each of the following sections (e.g., each section after the Tip section or portion, each section after the predetermined or set portion or section, etc.), and adjustments may be performed by a user (e.g., manually) and/or via one or more processors of the apparatus or system (e.g., automatically) to account for, or make adjustments or corrections to, unnecessary motion or motion that may be made more efficient. In one or more embodiments, adjustments or corrections may be performed automatically (e.g., by the device or system) and manually (e.g., by permitting manual adjustments by a user in addition to the automatic corrections or adjustments). In one or more embodiments, the adjustments may be stored in history along with the orientations such that the system or apparatus may perform the adjustments to each of the following sections such that a user may avoid having to repeatedly account for unnecessary motion manually because the system or apparatus performs or provides this feature or features.

One or more embodiments of the present disclosure may implement a direct path smoothing by having each stage position/state (or a position/state of another structure used for mapping path or path-like information) along a smoothing range gradually aim towards a final orientation. To make each stage position/state (or position/state of another structure used for mapping path or path-like information) along a smoothing range gradually aim towards a final orientation, a system or apparatus may have a “change in orientation” that occurs at a single stage position (or position/state of another structure used for mapping path or path-like information) be interpolated to or between steps, with each step being closer to the final orientation than the previous step. For example, in one or more non-limiting, non-exhaustive embodiment examples, a change in orientation may be represented by the change in the small vectors 900 as shown in FIG. 9 to have the motion be along a continuous direction as aforementioned.

In one or more embodiments of the present disclosure (and while not limited to only this definition), a “change of orientation” or a “change of state” (or a transition of state) may be defined in terms of direction and magnitude. For example, each interpolated step may have a same direction, and each interpolated step may have a larger magnitude as each step approaches a final orientation. Due to kinematics of one or more embodiments, any motion along a single direction may be the accumulation of a small motion in that direction. The small motion may have a unique or predetermined set of wire position or state changes to achieve the orientation change. Large or larger motion(s) in that direction may use a plurality of the small motions to achieve the large or larger motion(s). Dividing a large change into a series of multiple changes of the small or predetermined/set change may be used as one way to perform interpolation. Interpolation may be used in one or more embodiments to produce a desired or target motion, and at least one way to produce the desired or target motion may be to interpolate the change of wire positions or states.

In one or more embodiments, where smoothing is performed, interpolated orientations may be combined with values previously mapped to a corresponding stage position/state (or position/state of another structure used for mapping path or path-like information) (e.g., by simply adding the wire positions or states).

Due to kinematics of a robot, device, or system embodiment of the present disclosure, applying a same “change in position” or a “change in state” to two separate orientations/states may maintain a difference (e.g., a set difference, a predetermined difference, etc.) between the two separate orientations/states. Since an orientation/state difference may be defined as the difference between wire position/state in one or more embodiments (other embodiments are not limited thereto), changing both sets of wire positions or states by the same amount would not affect the orientation or state difference between the two separate orientations or states.

Orientations mapped to two subsequent stage positions/states (or positions/states of another structure used for mapping path or path-like information) may have a specific orientation difference between the orientations. In a case where smoothing is applied, the later (or second) stage position/state (or position/state of the another structure) has a same change in orientation that the earlier (or first) stage position/state (or position/state of the another structure) received such that the pose/state difference did not change. The smoothing process may include an additional step of a “small motion”, which operates to cause the pose/state difference to change by an amount of that small motion. Since the “small motion” operates to produce the same orientation/state change regardless of prior orientation/state, the small motion step operates to direct that orientation/state in a table towards a proper (e.g., set, desired, predetermined, selected, etc.) direction, while also maintaining a semblance or configuration of the prior path/state before the smoothing process was applied. Therefore, in one or more embodiments, it may be most efficient and effective to combine and compare wire positions or states to or with prior orientations or states while using a smoothing process to maintain the pre-existing orientation changes.

In one or more embodiments, a catheter or probe may transition, move, or adjust using a shortest possible volume. In a case where a following section or portion of the probe or catheter is being transitioned, moved, or adjusted, using the shortest possible volume my reduce or minimize an amount of disruption to positions or states of one or more (or all) of the distal/following sections or portions of the catheter or probe. In one or more embodiments, a process or algorithm may perform the transitioning, moving, or adjusting process more efficiently than computing a transformation stackup of each section or portion of the catheter or probe. Preferably, each interpolated step aims towards the final orientation in a desired direction such that any prior orientation which the interpolated step is combined with will also aim towards the desired direction to achieve the final orientation.

In one or more embodiments of the present disclosure, an apparatus or system may include one or more processors that operate to: receive or obtain an image or images showing pose or position (or other state) information of a tip section of a catheter or probe having a plurality of sections including at least the tip section; track a history of the pose or position (or other state) information of the tip section of the catheter or probe during a period of time; and use the history of the pose or position (or other state) information of the tip section to determine how to align or transition, move, or adjust (e.g., robotically, manually, automatically, etc.) each section of the plurality of sections of the catheter or probe.

In one or more embodiments, one or more additional image or images may be received or obtained to show the catheter or probe after each section of the plurality of sections of the catheter or probe has been aligned or adjusted (e.g., robotically, manually, automatically, etc.) based on the history of the pose or position (or other state) information of the tip section. In one or more embodiments, the apparatus or system may include a display to display the image or images showing the aligned or adjusted sections of the catheter or probe. In one or more embodiments, the pose or position (or other state) information may include, but is not limited to, a target pose or position (or other state) or a final pose or position (or other state) that the tip section is set to reach, an interpolated pose or position (or other state) of the tip section (e.g., an interpolation of the tip section between two positions or poses (or other states) (e.g., between pose or position (or other state) A to pose or position (or other state) B) where the apparatus or system sends pose (or other state) change information in steps based on a desired, set, or predetermined speed; between poses or positions where each pose or position (or other state) of the catheter or probe takes or is disposed is tracked during the transition; etc.), and a measured pose or position (or other state) (e.g., using tracked poses or positions (or other states), using encoder positions (or other states) of each wire motor, etc.) where the one or more processors may further operate to calculate or derive a current position or position (or state) that a section (e.g., the tip section, one of the other sections of the plurality of sections of the probe or catheter, etc.) of the probe or catheter is taking. In addition to using one or more types of poses or positions (or other states), each pose or position (or state) may be converted (e.g., via the one or more processors) between the following formats: Drive/Driving Wire Positions (or state(s)); and/or Coordinates (three-dimensional (3D) Position and Orientation (or other state(s))).

In one or more embodiments, an apparatus or system may include a camera deployed at a tip of a catheter or probe and may be bent with the catheter or probe, and/or the camera may be detachably attached to, or removably inserted into, the steerable catheter or probe. In one or more embodiments, an apparatus or system may include a display controller, or the one or more processors may display the image or images for display on a display.

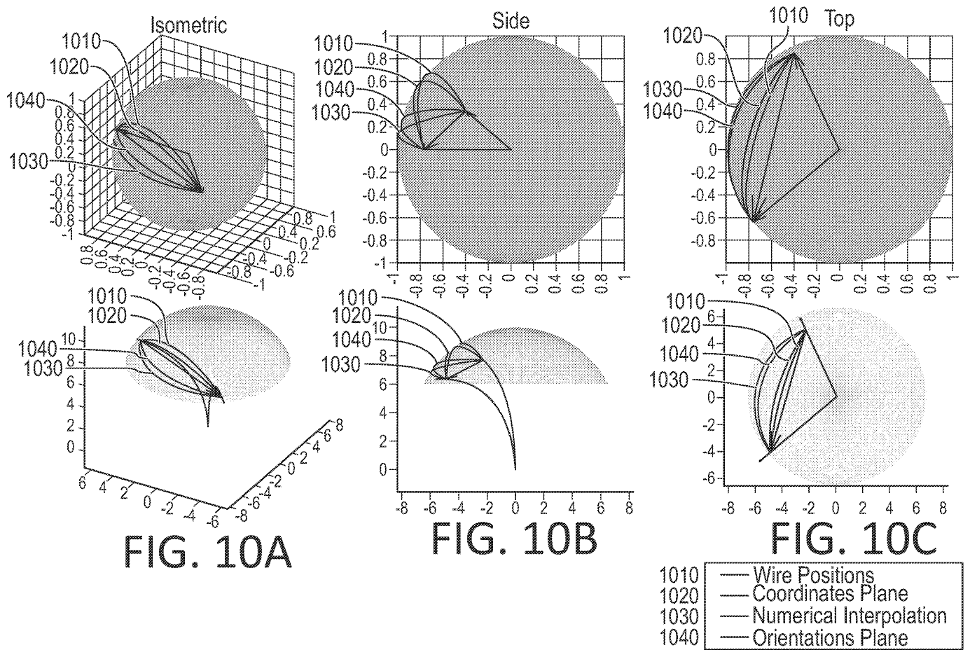

FIGS. 10A-10C show three (top) orientation vector trace graphs for isometric, side, and top information, respectively, and show three (bottom) end coordinates trace graphs for isometric, side, and top information, respectively, for different paths in accordance with one or more aspects of the present disclosure. As shown in each view, reference number 1010 shows a path 1010 for interpolating between wire positions. A pose transition keeps end coordinates on a same coordinates plane 1020 defined by a base of the section or portion of the catheter or probe and the end coordinates of the Start and End poses/positions/states/etc. A numerical interpolation curve 1030 (which may correspond to curve or path 800 discussed above in one or more embodiments) may operate to use transitioning based on numerical interpolation between Start and End pose plane and angle values. An orientations plane 1040 curve may show how a pose transition keeps an orientation vector on a same plane defined by the orientation vectors of the Start and End poses/positions/states/etc. FIGS. 10A-10C show a scenario where one or more methods, processes, or algorithms of the present disclosure may create different traces. The top diagrams or graphs of FIGS. 10A-10C show the trace of each orientation vector during a pose/position/state transition. The bottom diagrams or graphs of FIGS. 10A-10C show the trace of each End coordinate during the pose/position/state transition.

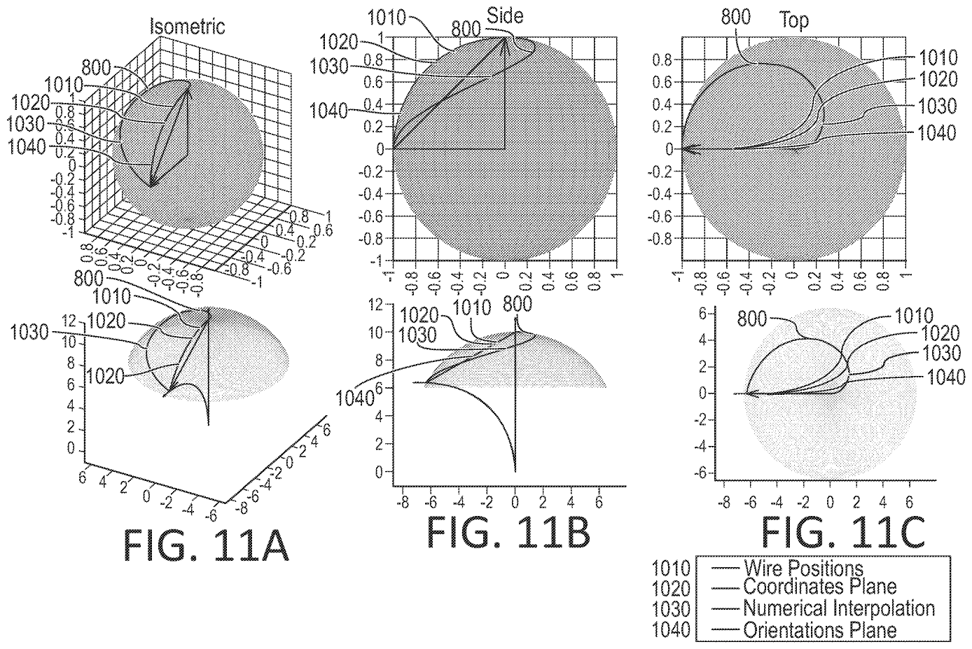

FIGS. 11A-11C show three (top graphs) orientation vector trace graphs for isometric, side, and top information, respectively, and show three (bottom graphs) end coordinates trace graphs for isometric, side, and top information, respectively, for a same or similar path in accordance with one or more aspects of the present disclosure. As shown in each view, reference number 1010 shows a path 1010 for interpolating between wire positions. A pose transition keeps end coordinates on a same coordinates plane 1020 defined by a base of the section or portion of the catheter or probe and the end coordinates of the Start and End poses/positions/states/etc. A numerical interpolation curve 1030 (which may correspond to curve or path 800 discussed above in one or more embodiments) may operate to use transitioning based on numerical interpolation between Start and End pose plane and angle values. An orientations plane 1040 curve may show how a pose transition keeps an orientation vector on a same plane defined by the orientation vectors of the Start and End poses/positions/states/etc. FIGS. 11A-11C show a scenario where one or more methods, processes, or algorithms of the present disclosure may create same or similar traces or path(s) for the paths 1010, 1020, and 1040. The top diagrams or graphs of FIGS. 11A-11C show the trace of each orientation vector during a pose/position/state transition. The bottom diagrams or graphs of FIGS. 11A-11C show the trace of each End coordinate during the pose/position/state transition.

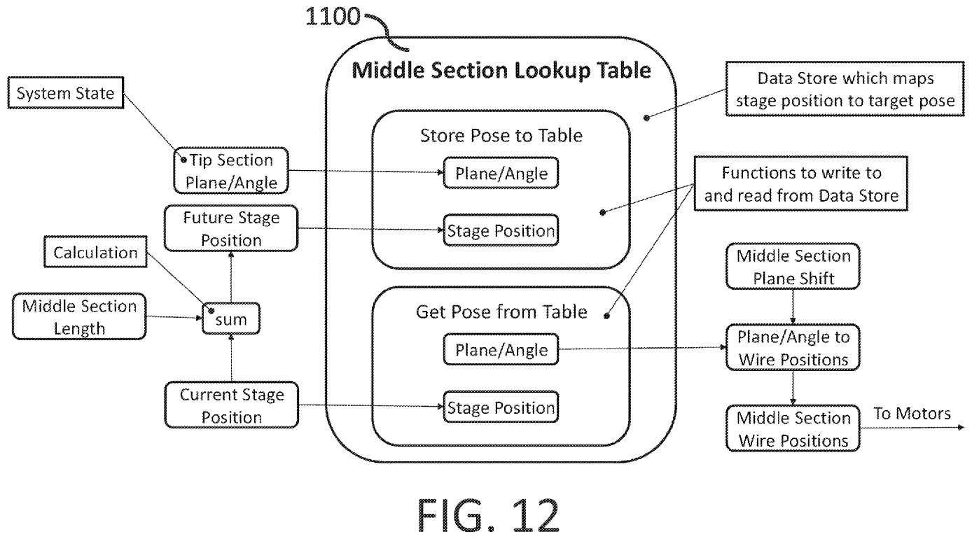

One or more embodiments of the present disclosure may perform one or more workflows using one or more features as shown in, but not limited to, the embodiments of FIGS. 12-14. For example, one or more embodiments may store information in and retrieve information from a look up table, such as, but not limited to, a middle section lookup table 1100 as shown in FIGS. 12-14.

As shown in FIG. 12, a device, system, method, or storage medium of the present disclosure state may obtain or determine a Tip Section plane/angle and a current stage position/state (or position/state of another structure used for mapping path or path-like information), and that information (e.g., state information for a section or portion, pose or position information for a section or portion, stage position/state information, position/state information for the another structure, etc.) may be stored to a table or tables. A future (or other same/similar/approximate/corresponding) stage position or state may be determined from a current stage position or state by performing a calculation or sum based on middle section length data, and the determined future (or other same/similar/approximate/corresponding) stage position or state may be stored in a table. For example, a Middle (or other) section or portion may replicate or copy an orientation or state of a predetermined or set section or portion (e.g., a Tip section or portion, another selected section or portion, etc.) in a case where the Middle (or other) section or portion reaches that future (or other same/similar/approximate/corresponding) stage position or state (or a position or state of another structure used to map a path or path-like information). Functions or steps may be performed by one or more processors to write and read information from Data Store, and Data Store may map stage position/state (a position/state of another structure used to map a path or path-like information) to a target pose or position (or other state). One or more embodiments may determine how to adjust one or more wire positions (e.g., for the drive or driving wires, such as, but not limited to, the wires 142, 144, 146, etc. discussed above) based on a middle section plane shift and based on plane/angle information such that the wire positions or states for the middle section may be determined and such information may be sent to the motors (e.g., wire driving motors) to make the adjustment(s), change(s), state, or smoothing operation(s). In one or more embodiments, positions or states for drive/driving wires may not be used for performing interpolation and/or any other techniques discussed herein, and other means of controlling an orientation, position, state, etc. of a section or portion of a catheter or probe may be used, such as, but not limited to, pneumatic actuators.

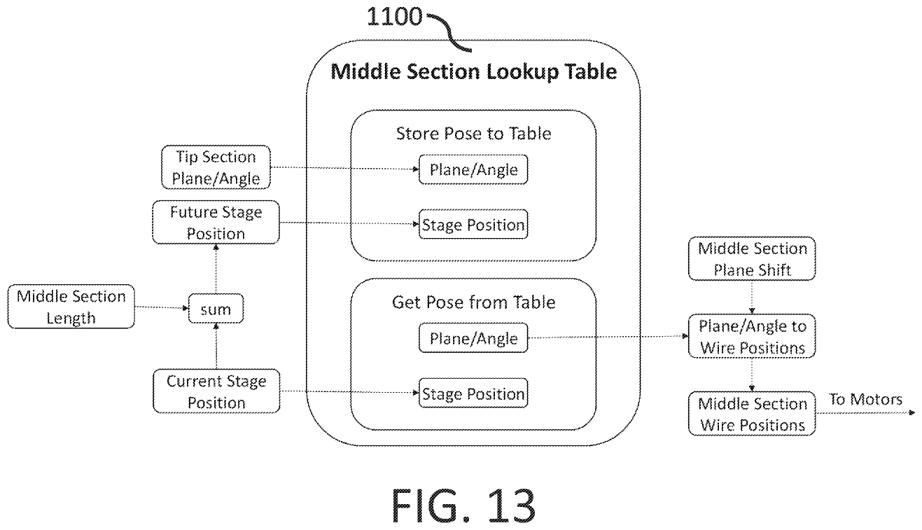

One or more embodiments may align a middle pose to a tip pose as shown in FIG. 13. A device, system, method, or storage medium of the present disclosure state may obtain or determine a Tip Section plane/angle and a current stage position or state, and that information (e.g., pose or position (or other state) information for a section or portion, stage position or other state information, etc.) may be stored to a table or tables. A future stage position or state (or other same/similar/approximate/corresponding position or state) may be determined from a current stage position or state by performing a sum based on middle section length data, and the determined future stage position or state (or other same/similar/approximate/corresponding position or state) may be stored in a table. One or more embodiments may determine how to adjust one or more wire positions or states (e.g., for the drive or driving wires, such as, but not limited to, the wires 142, 144, 146, etc. discussed above) based on a middle section plane shift and based on plane/angle (or other state) information such that the wire positions for the middle section may be determined and such information may be sent to the motors (e.g., wire driving motors, pneumatic actuators, etc.) to make the adjustment(s), change(s), state transition(s), or smoothing operation(s) to align the middle pose or position (or state) to the Tip Section or portion (or other predetermined or selected section or portion). In one or more embodiments, the calculation shown in FIG. 10 for the sum of the future stage position or state (or other same/similar/approximate/corresponding stage position or state) may be omitted, the system stage (or other structure used for mapping a path or path-like information) feature, and the Data Store features may be omitted. In one or more embodiments, a state of a portion or section may be stored as a reference and/or may come from a user-initiated state change occurring during retraction, in addition to insertion and to being stationary.

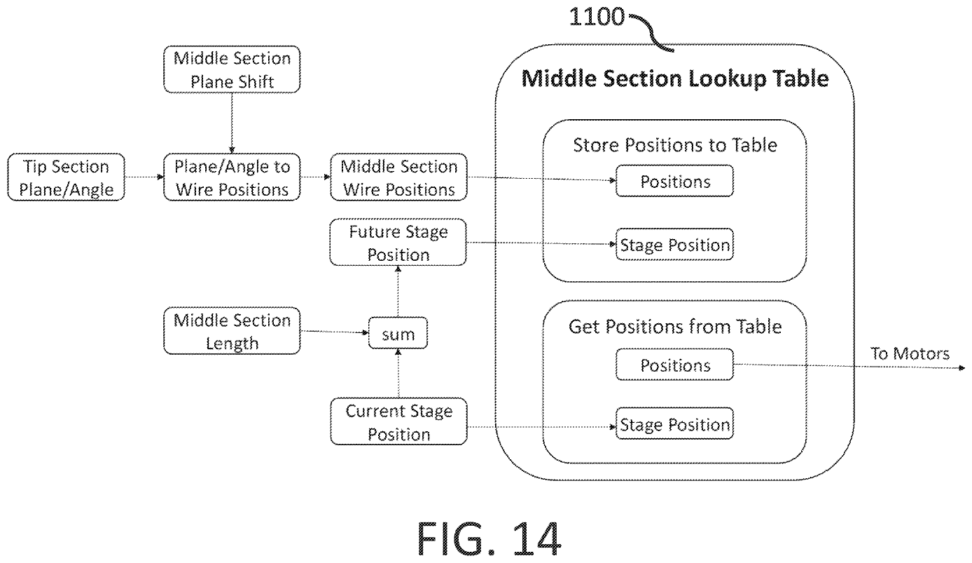

One or more additional embodiments of the present disclosure may align a middle pose or state to a tip pose or state as shown in FIG. 14. As shown in FIG. 14, middle section wire positions or states may be determined and/or adjusted based on a Tip Section (or other predetermined or chosen portion or section) plane and/or angle information, based on a Middle Section plane shift, and based on application of the plane and/or angle (and/or other state) information to the wire position(s) or state(s), and the middle section wire positions or states may be stored in a table or section of the Middle Section Lookup Table 1100. A future stage position or state (or other same/similar/approximate/corresponding position or state) may be determined from a current stage position or state (or a position or state of another structure used for mapping a path or path-like information) by performing a sum based on middle section length data, and the current stage (or other structure) position or state information and/or the determined future stage (or other structure) position or state (or other same/similar/approximate/corresponding position or state) information may be stored in a table or tables (or different sections) of the Middle Section Lookup Table 1100. In one or more embodiments, a Middle (or other) section or portion may replicate or copy an orientation or state of a predetermined or set section or portion (e.g., a Tip section or portion, another selected section or portion, etc.) in a case where the Middle (or other) section or portion reaches that future stage position or state (or a position or state of another structure used to map a path or path-like information; or other same/similar/approximate/corresponding position or state; etc.). The position or state information (e.g., the current and/or future stage (or other structure) position or state information, other same/similar/approximate/corresponding position or state information, the middle section wire positions or states information, any other state(s) discussed herein, etc.) may be obtained from the Middle Section Lookup Table 1100 and sent to the motors (e.g., wire driving motors) to make the adjustment(s), change(s), state transition(s), or smoothing operation(s) to align the middle pose or position (or other state) to the Tip Section or portion (or other predetermined or selected section or portion). In one or more embodiments, as aforementioned, any other predetermined or chosen portion or section (e.g., other than the Tip section or portion) may be used as a source for a target state for another portion or section of the catheter or probe. This is particularly useful in a Targeting mode, where a user or one or more processors may change a pose or state of a Middle section or portion and the user or one or more processors operate to include that change in the motion or state changes/transitions of the Proximal or Tip section or portion (or any other desired or selected section or portion) of a catheter or probe. Additionally or alternatively, different portions or sections may use different portions or sections as a source for making such changes (e.g., a middle portion or section may use a tip section or portion, a subsequent portion or section may use the middle portion or section, etc.). In one or more embodiments, a source may change throughout or during the procedure or techniques being performed for such changes. A source change may occur due to one or more factors, such as, but not limited to, different modes, scenarios, settings, etc.

Alternatively or additionally, in addition to finding a direct (or approximate) orientation or state transition, one or more embodiments may include a direct (or approximate) end effector position or state transition and/or may include a continuous (or specific) local pose or state change during transitioning. In one or more embodiments using drive or driving wire interpolation, a device, system, method, or storage medium may use such transitioning techniques as desired. For example, a user may instruct or make state transitions or changes using a joystick (e.g., by holding the joystick in a constant direction) as discussed herein, or one or more processors may automatically control (or control in response to a request) such state transitions or changes.

One or more features discussed herein may be used for performing correction, adjustment, and/or smoothing (e.g., direct FTL smoothing, path smoothing, continuum robot smoothing, etc.). FIG. 15 is a flowchart showing steps of at least one procedure for performing correction, adjustment, and/or smoothing of a continuum robot/catheter device (e.g., such as continuum robot/catheter device 104). One or more of the processors discussed herein may execute the steps shown in FIG. 15, and these steps may be performed by executing a software program read from a storage medium, including, but not limited to, the ROM 110 or HDD 150, by CPU 120 or by any other processor discussed herein. One or more methods of performing correction, adjustment, and/or smoothing (e.g., direct FTL smoothing) for a catheter or probe of a continuum robot device or system may include one or more of the following steps: (i) in step S1300, instructing a distal bending section or portion of a catheter or a probe of a continuum robot such that the distal bending section or portion achieves, or is disposed at, a bending pose or position; (ii) in step S1301, storing or obtaining the bending pose or position of the distal bending section or portion and storing or obtaining a position of a motorized linear stage that operates to move the catheter or probe of the continuum robot in a case where a forward motion, or a motion in a set or predetermined direction or directions, of the motorized linear stage is instructed or commanded; (iii) in step S1302, generating a goal or target bending pose or position (or other state) for each corresponding section or portion of the catheter or probe from, or based on, the previous bending section or portion or based on a previous pose or state of a Distal bending section or portion; (iv) in step S1303, generating interpolated poses or positions for each of the sections or portions of the catheter or probe between the respective goal or target bending pose or position and a respective current bending pose or position of each of the sections or portions of the catheter or probe, wherein the interpolated poses or positions are generated such that an orientation vector of the interpolated poses or positions are on a plane that an orientation vector of the respective goal or target bending pose or position and an orientation vector of a respective current bending pose or position create or define; and/or (v) in step S1304, instructing or commanding each of the sections or portions of the catheter or probe to move to or be disposed at the respective interpolated poses or positions during the forward motion, or the motion in the set or predetermined direction, of the previous section(s) or portion(s) of the catheter or probe (see FIG. 15).

In one or more embodiments, a non-transitory computer-readable storage medium may store at least one program for causing a computer to execute a method for performing correction, adjustment, and/or smoothing (e.g., direct FTL smoothing), the method comprising one or more of the following steps: (i) in step S1300, instructing a distal bending section or portion of a catheter or a probe of a continuum robot such that the distal bending section or portion achieves, or is disposed at, a bending pose or position; (ii) in step S1301, storing or obtaining the bending pose or position of the distal bending section or portion and storing or obtaining a position of a motorized linear stage that operates to move the catheter or probe of the continuum robot in a case where a forward motion, or a motion in a set or predetermined direction or directions, of the motorized linear stage is instructed or commanded; (iii) in step S1302, generating a goal or target bending pose or position for each corresponding section or portion of the catheter or probe from, or based on, the previous bending section or portion; (iv) in step S1303, generating interpolated poses or positions for each of the sections or portions of the catheter or probe between the respective goal or target bending pose or position and a respective current bending pose or position of each of the sections or portions of the catheter or probe, wherein the interpolated poses or positions are generated such that an orientation vector of the interpolated poses or positions are on a plane that an orientation vector of the respective goal or target bending pose or position and an orientation vector of a respective current bending pose or position create or define; and/or (v) in step S1304, instructing or commanding each of the sections or portions of the catheter or probe to move to or be disposed at the respective interpolated poses or positions during the forward motion, or the motion in the set or predetermined direction, of the previous section(s) or portion(s) of the catheter or probe (see FIG. 15).

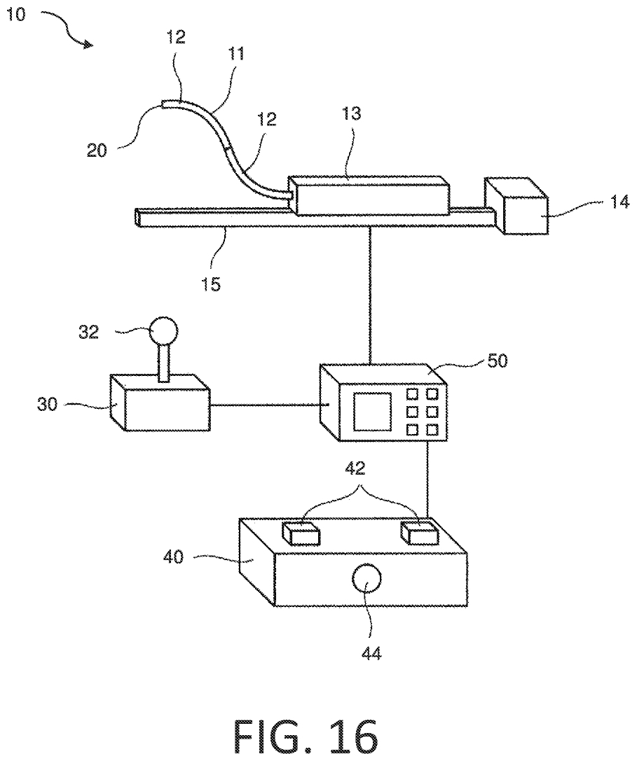

One or more of the aforementioned features may be used with a continuum robot and related features as disclosed in U.S. Provisional Pat. App. No. 63/150,859, filed on Feb. 18, 2021, the disclosure of which is incorporated by reference herein in its entirety. For example, FIGS. 16 to 18 illustrate features of at least one embodiment of a continuum robot apparatus 10 configuration to implement automatic correction of a direction to which a tool channel or a camera moves or is bent in a case where a displayed image is rotated. The continuum robot apparatus 10 enables to keep a correspondence between a direction on a monitor (top, bottom, right or left of the monitor) and a direction the tool channel or the camera moves on the monitor according to a particular directional command (up, down, turn right or turn left) even if the displayed image is rotated.

As shown in FIGS. 16 and 17, the continuum robot apparatus 10 may include one or more of a continuum robot 11, an image capture unit 20, an input unit 30, a guide unit 40, a controller 50, and a display 60. The image capture unit 20 may be a camera or other image capturing device. The continuum robot 11 may include one or more flexible portions 12 connected together and configured so that the one or more flexible portions 12 may be curved or rotated about in different directions. The continuum robot 11 may include a drive unit 13, a movement drive unit 14, and a linear drive or guide 15. The movement drive unit 14 operates to cause the drive unit 13 to move along the linear drive or guide 15.

The input unit 30 has an input element 32 and is configured to allow a user to positionally adjust the flexible portions 12 of the continuum robot 11. The input unit 30 may be configured as a mouse, a keyboard, joystick, lever, or another shape to facilitate user interaction. The user may provide an operation input through the input element 32, and the continuum robot apparatus 10 may receive information of the input element 32 and one or more input/output devices, which may include, but are not limited to, a receiver, a transmitter, a speaker, a display, an imaging sensor, a user input device, which may include a keyboard, a keypad, a mouse, a position tracked stylus, a position tracked probe, a foot switch, a microphone, etc. The guide unit 40 is a device that includes one or more buttons, knobs, switches, etc. 42, 44, that a user may use to adjust various parameters of the continuum robot 10, such as the speed (e.g., rotational speed, translational speed, etc.), angle or plane, or other parameters.

FIG. 18 illustrates at least one embodiment of a controller 50 according to one or more features of the present disclosure. The controller 50 may be configured to control the elements of the continuum robot apparatus 10 and has one or more of a CPU 51, a memory 52, a storage 53, an input and output (I/O) interface 54, and communication interface 55. The continuum robot apparatus 10 may be interconnected with medical instruments or a variety of other devices, and may be controlled independently, externally, or remotely by the controller 50. In one or more embodiments of the present disclosure, one or more features of the continuum robot apparatus 10 and one or more features of the continuum robot or catheter or probe system 1000 may be used in combination or alternatively to each other.

The memory 52 may be used as a work memory or may include any memory discussed in the present disclosure. The storage 53 stores software or computer instructions, and may be any type of storage, data storage 150, or other memory or storage discussed in the present disclosure. The CPU 51, which may include one or more processors, circuitry, or a combination thereof, executes the software developed in the memory 52 (or any other memory discussed herein). The I/O interface 54 operates to input information from the continuum robot apparatus 10 to the controller 50 and to output information for displaying to the display 60 (or any other display discussed herein, such as, but not limited to, display 1209 discussed below).

The communication interface 55 may be configured as a circuit or other device for communicating with components included in the apparatus 10, and with various external apparatuses connected to the apparatus via a network. For example, the communication interface 55 may store information to be output in a transfer packet and may output the transfer packet to an external apparatus via the network by communication technology such as Transmission Control Protocol/Internet Protocol (TCP/IP). The apparatus may include a plurality of communication circuits according to a desired communication form.

The controller 50 may be communicatively interconnected or interfaced with one or more external devices including, for example, one or more data storages (e.g., the data storage 150, the SSD or storage drive 1207 discussed below, or any other storage discussed herein), one or more external user input/output devices, or the like. The controller 50 may interface with other elements including, for example, one or more of an external storage, a display, a keyboard, a mouse, a sensor, a microphone, a speaker, a projector, a scanner, a display, an illumination device, etc.

The display 60 may be a display device configured, for example, as a monitor, an LCD (liquid panel display), an LED display, an OLED (organic LED) display, a plasma display, an organic electro luminescence panel, or any other display discussed herein. Based on the control of the apparatus, a screen may be displayed on the display 60 showing one or more images, such as, but not limited to, one or more images being captured, captured images, captured moving images recorded on the storage unit, etc.

The components may be connected together by a bus 56 so that the components may communicate with each other. The bus 56 transmits and receives data between these pieces of hardware connected together, or the bus 56 transmits a command from the CPU 51 to the other pieces of hardware. The components may be implemented by one or more physical devices that may be coupled to the CPU 51 through a communication channel. For example, the controller 50 may be implemented using circuitry in the form of ASIC (application specific integrated circuits) or other similar circuits as discussed herein. Alternatively, the controller 50 may be implemented as a combination of hardware and software, where the software is loaded into a processor from a memory or over a network connection. Functionality of the controller 50 may be stored on a storage medium, which may include, but is not limited to, RAM (random-access memory), magnetic or optical drive, diskette, cloud storage, etc.

The units described throughout the present disclosure are exemplary and/or preferable modules for implementing processes described in the present disclosure. However, one or mor embodiments of the present disclosure are not limited thereto. The term “unit”, as used herein, may generally refer to firmware, software, hardware, or other component, such as circuitry or the like, or any combination thereof, that is used to effectuate a purpose. The modules may be hardware units (such as circuitry, firmware, a field programmable gate array, a digital signal processor, an application specific integrated circuit or the like) and/or software modules (such as a computer readable program, instructions stored in a memory or storage medium, etc.). The modules for implementing the various steps are not described exhaustively above. However, where there is a step of performing a certain process, there may be a corresponding functional module or unit (implemented by hardware and/or software) for implementing the same process. Technical solutions by all combinations of steps described and units corresponding to these steps are included in the present disclosure.

One or more adjustment, correction, and/or smoothing features of the present disclosure may be used with one or more image correction or adjustment features in one or more embodiments. One or more adjustments, corrections, or smoothing functions for a catheter or probe device and/or a continuum robot may adjust a path of one or more sections or portions of the catheter or probe device and/or the continuum robot (e.g., the continuum robot 104, the continuum robot device 10, etc.), and one or more embodiments may make a corresponding adjustment or correction to an image view. For example, in one or more embodiments the medical tool may be a bronchoscope.

While one or more features of the present disclosure have been described with reference to exemplary embodiments, it is to be understood that the present disclosure is not limited to the disclosed exemplary embodiments. The scope of the claims is to be accorded the broadest interpretation so as to encompass all modifications, equivalent structures, and functions.

A computer, such as the console or computer 1200, 1200′, may perform any of the steps, processes, and/or techniques discussed herein for any apparatus and/or system being manufactured or used, any of the embodiments shown in FIGS. 1-20, any other apparatus or system discussed herein, etc.

There are many ways to control a continuum robot, correct or adjust an image or a path (or one or more sections or portions of) a continuum robot (or other probe or catheter device or system), or perform any other measurement or process discussed herein, to perform continuum robot method(s) or algorithm(s), and/or to control at least one continuum robot device/apparatus, system and/or storage medium, digital as well as analog. In at least one embodiment, a computer, such as the console or computer 1200, 1200′, may be dedicated to control and/or use continuum robot devices, systems, methods, and/or storage mediums for use therewith described herein.

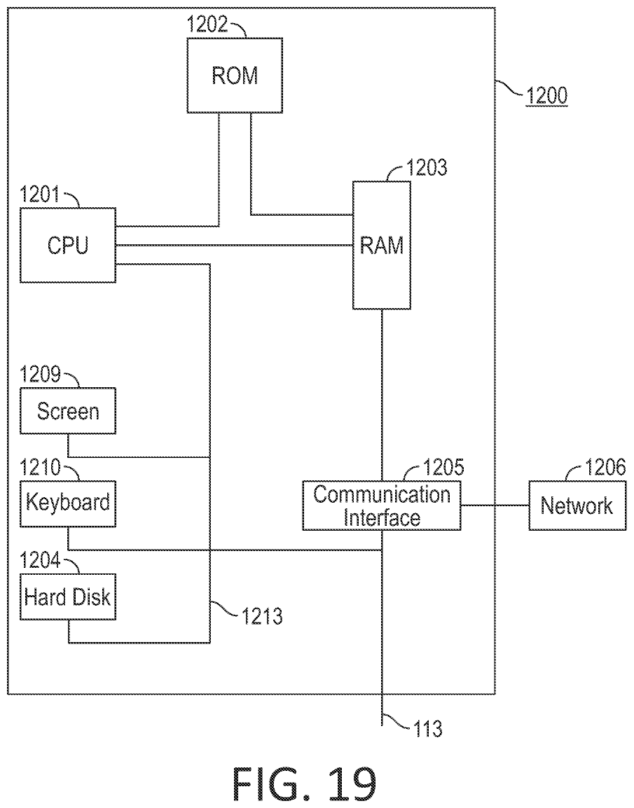

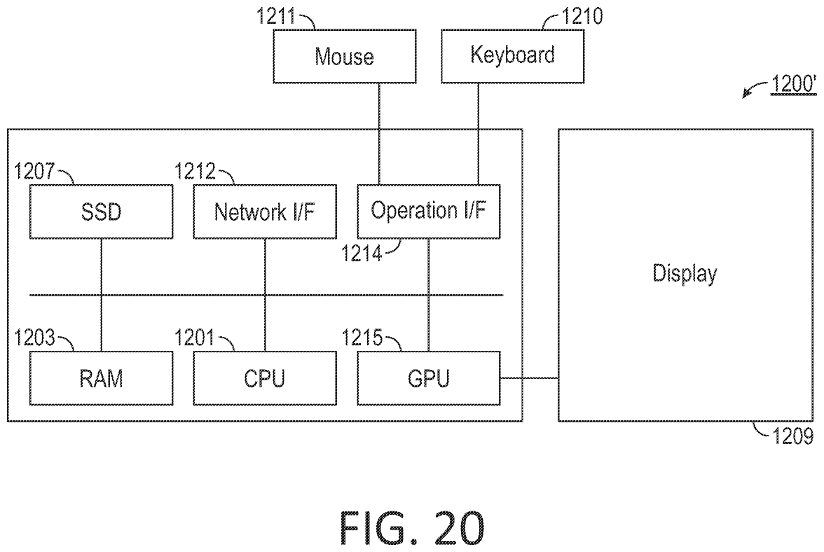

The one or more detectors, sensors, cameras, or other components of the apparatus or system embodiments (e.g. of the system 1000 of FIG. 1 or any other system discussed herein) may transmit the digital or analog signals to a processor or a computer such as, but not limited to, an image processor or display controller 100, a controller 102, a CPU 120, a controller 50, a CPU 51, a processor or computer 1200, 1200′ (see e.g., at least FIGS. 1-5, 16-18, and 19-20), a combination thereof, etc. The image processor may be a dedicated image processor or a general purpose processor that is configured to process images. In at least one embodiment, the computer 1200, 1200′ may be used in place of, or in addition to, the image processor or display controller 100 and/or the controller 102 (or any other processor or controller discussed herein, such as, but not limited to, the controller 50, the CPU 51, etc.). In an alternative embodiment, the image processor may include an ADC and receive analog signals from the one or more detectors or sensors of the system 1000 (or any other system discussed herein). The image processor may include one or more of a CPU, DSP, FPGA, ASIC, or some other processing circuitry. The image processor may include memory for storing image, data, and instructions. The image processor may generate one or more images based on the information provided by the one or more detectors, sensors, or cameras. A computer or processor discussed herein, such as, but not limited to, a processor of the devices, apparatuses or systems of FIGS. 1-5 and 16-18, the computer 1200, the computer 1200′, the image processor, etc. may also include one or more components further discussed herein below (see e.g., FIGS. 19-20).

Electrical analog signals obtained from the output of the system 1000 or the components thereof, and/or from the devices, apparatuses, or systems of FIGS. 1-5 and 16-18, may be converted to digital signals to be analyzed with a computer, such as, but not limited to, the computers or controllers 100, 102 of FIG. 1, the computer 1200, 1200′, etc.

As aforementioned, there are many ways to control a continuum robot, correct or adjust an image, correct, adjust, or smooth a path (or section or portion) of a continuum robot, or perform any other measurement or process discussed herein, to perform continuum robot method(s) or algorithm(s), and/or to control at least one continuum robot device/apparatus, system and/or storage medium, digital as well as analog. By way of a further example, in at least one embodiment, a computer, such as the computer or controllers 100, 102 of FIG. 1, the console or computer 1200, 1200′, etc., may be dedicated to the control and the monitoring of the continuum robot devices, systems, methods and/or storage mediums described herein.

The electric signals used for imaging may be sent to one or more processors, such as, but not limited to, the processors or controllers 100, 102 of FIGS. 1-5, a computer 1200 (see e.g., FIG. 19), a computer 1200′ (see e.g., FIG. 20), etc. as discussed further below, via cable(s) or wire(s), such as, but not limited to, the cable(s) or wire(s) 113 (see FIG. 19). Additionally or alternatively, the computers or processors discussed herein are interchangeable, and may operate to perform any of the feature(s) and method(s) discussed herein.

Various components of a computer system 1200 (see e.g., the console or computer 1200 as may be used as one embodiment example of the computer, processor, or controllers 100, 102 shown in FIG. 1) are provided in FIG. 19. A computer system 1200 may include a central processing unit (“CPU”) 1201, a ROM 1202, a RAM 1203, a communication interface 1205, a hard disk (and/or other storage device) 1204, a screen (or monitor interface) 1209, a keyboard (or input interface; may also include a mouse or other input device in addition to the keyboard) 1210 and a BUS (or “Bus”) or other connection lines (e.g., connection line 1213) between one or more of the aforementioned components (e.g., as shown in FIG. 19). In addition, the computer system 1200 may comprise one or more of the aforementioned components. For example, a computer system 1200 may include a CPU 1201, a RAM 1203, an input/output (I/O) interface (such as the communication interface 1205) and a bus (which may include one or more lines 1213 as a communication system between components of the computer system 1200; in one or more embodiments, the computer system 1200 and at least the CPU 1201 thereof may communicate with the one or more aforementioned components of a continuum robot device or system using same, such as, but not limited to, the system 1000, the devices/systems of FIGS. 1-5, and/or the systems/apparatuses of FIGS. 16-18, discussed herein above, via one or more lines 1213), and one or more other computer systems 1200 may include one or more combinations of the other aforementioned components (e.g., the one or more lines 1213 of the computer 1200 may connect to other components via line 113). The CPU 1201 is configured to read and perform computer-executable instructions stored in a storage medium. The computer-executable instructions may include those for the performance of the methods and/or calculations described herein. The computer system 1200 may include one or more additional processors in addition to CPU 1201, and such processors, including the CPU 1201, may be used for controlling and/or manufacturing a device, system or storage medium for use with same or for use with any continuum robot technique(s), and/or use with image correction or adjustment technique(s) discussed herein. The system 1200 may further include one or more processors connected via a network connection (e.g., via network 1206). The CPU 1201 and any additional processor being used by the system 1200 may be located in the same telecom network or in different telecom networks (e.g., performing, manufacturing, controlling, calculation, and/or using technique(s) may be controlled remotely).

The I/O or communication interface 1205 provides communication interfaces to input and output devices, which may include the one or more of the aforementioned components of any of the systems discussed herein (e.g., the controller 100, the controller 102, the displays 101-1, 101-2, the actuator 103, the continuum device 104, the operating portion or controller 105, the EM tracking sensor 106, the position detector 107, the rail 108, etc.), a microphone, a communication cable and a network (either wired or wireless), a keyboard 1210, a mouse (see e.g., the mouse 1211 as shown in FIG. 20), a touch screen or screen 1209, a light pen and so on. The communication interface of the computer 1200 may connect to other components discussed herein via line 113 (as diagrammatically shown in FIG. 19). The Monitor interface or screen 1209 provides communication interfaces thereto.

Any methods and/or data of the present disclosure, such as, but not limited to, the methods for using and/or controlling a continuum robot or catheter device, system, or storage medium for use with same and/or method(s) for imaging, performing tissue or sample characterization or analysis, performing diagnosis, planning and/or examination, for performing adjustment, correction, or smoothing technique(s) (e.g., to a path of, to a pose or position of, or to one or more sections or portions of, a continuum robot, a catheter or a probe), and/or for performing image correction or adjustment technique(s), as discussed herein, may be stored on a computer-readable storage medium. A computer-readable and/or writable storage medium used commonly, such as, but not limited to, one or more of a hard disk (e.g., the hard disk 1204, a magnetic disk, etc.), a flash memory, a CD, an optical disc (e.g., a compact disc (“CD”) a digital versatile disc (“DVD”), a Blu-ray™ disc, etc.), a magneto-optical disk, a random-access memory (“RAM”) (such as the RAM 1203), a DRAM, a read only memory (“ROM”), a storage of distributed computing systems, a memory card, or the like (e.g., other semiconductor memory, such as, but not limited to, a non-volatile memory card, a solid state drive (SSD) (see SSD 1207 in FIG. 20), SRAM, etc.), an optional combination thereof, a server/database, etc. may be used to cause a processor, such as, the processor or CPU 1201 of the aforementioned computer system 1200 to perform the steps of the methods disclosed herein. The computer-readable storage medium may be a non-transitory computer-readable medium, and/or the computer-readable medium may comprise all computer-readable media, with the sole exception being a transitory, propagating signal in one or more embodiments. The computer-readable storage medium may include media that store information for predetermined, limited, or short period(s) of time and/or only in the presence of power, such as, but not limited to Random Access Memory (RAM), register memory, processor cache(s), etc. Embodiment(s) of the present disclosure may also be realized by a computer of a system or apparatus that reads out and executes computer executable instructions (e.g., one or more programs) recorded on a storage medium (which may also be referred to more fully as a “non-transitory computer-readable storage medium”) to perform the functions of one or more of the above-described embodiment(s) and/or that includes one or more circuits (e.g., application specific integrated circuit (ASIC)) for performing the functions of one or more of the above-described embodiment(s), and by a method performed by the computer of the system or apparatus by, for example, reading out and executing the computer executable instructions from the storage medium to perform the functions of one or more of the above-described embodiment(s) and/or controlling the one or more circuits to perform the functions of one or more of the above-described embodiment(s).

In accordance with at least one aspect of the present disclosure, the methods, devices, systems, and computer-readable storage mediums related to the processors, such as, but not limited to, the processor of the aforementioned computer 1200, the processor of computer 1200′, the controller 100, the controller 102, etc., as described above may be achieved utilizing suitable hardware, such as that illustrated in the figures. Functionality of one or more aspects of the present disclosure may be achieved utilizing suitable hardware, such as that illustrated in FIG. 19. Such hardware may be implemented utilizing any of the known technologies, such as standard digital circuitry, any of the known processors that are operable to execute software and/or firmware programs, one or more programmable digital devices or systems, such as programmable read only memories (PROMs), programmable array logic devices (PALs), etc. The CPU 1201 (as shown in FIG. 19 or FIG. 20, and/or which may be included in the computer, processor, controller and/or CPU 120 of FIGS. 1-5), CPU 51, and/or the CPU 120 may also include and/or be made of one or more microprocessors, nanoprocessors, one or more graphics processing units (“GPUs”; also called a visual processing unit (“VPU”)), one or more Field Programmable Gate Arrays (“FPGAs”), or other types of processing components (e.g., application specific integrated circuit(s) (ASIC)). Still further, the various aspects of the present disclosure may be implemented by way of software and/or firmware program(s) that may be stored on suitable storage medium (e.g., computer-readable storage medium, hard drive, etc.) or media (such as floppy disk(s), memory chip(s), etc.) for transportability and/or distribution. The computer may include a network of separate computers or separate processors to read out and execute the computer executable instructions. The computer executable instructions may be provided to the computer, for example, from a network or the storage medium. The computers or processors (e.g., 100, 102, 120, 50, 51, 1200, 1200′, etc.) may include the aforementioned CPU structure, or may be connected to such CPU structure for communication therewith.

As aforementioned, hardware structure of an alternative embodiment of a computer or console 1200′ is shown in FIG. 20. The computer 1200′ includes a central processing unit (CPU) 1201, a graphical processing unit (GPU) 1215, a random access memory (RAM) 1203, a network interface device 1212, an operation interface 1214 such as a universal serial bus (USB) and a memory such as a hard disk drive or a solid-state drive (SSD) 1207. Preferably, the computer or console 1200′ includes a display 1209 (and/or the displays 101-1, 101-2). The computer 1200′ may connect with one or more components of a system (e.g., the systems/apparatuses of FIGS. 1-5, 16-18, etc.) via the operation interface 1214 or the network interface 1212. The operation interface 1214 is connected with an operation unit such as a mouse device 1211, a keyboard 1210 or a touch panel device. The computer 1200′ may include two or more of each component. Alternatively, the CPU 1201 or the GPU 1215 may be replaced by the field-programmable gate array (FPGA), the application-specific integrated circuit (ASIC) or other processing unit depending on the design of a computer, such as the computer 1200, the computer 1200′, etc.

At least one computer program is stored in the SSD 1207 (or any other storage device or drive discussed herein), and the CPU 1201 loads the at least one program onto the RAM 1203, and executes the instructions in the at least one program to perform one or more processes described herein, as well as the basic input, output, calculation, memory writing, and memory reading processes. The computer, such as the computer 1200, 1200′, the computer, processors, and/or controllers of FIGS. 1-5 and/or FIGS. 16-18, etc., communicates with the one or more components of the apparatuses/systems of FIGS. 1-5, of FIGS. 16-18, and/or of any other system(s) discussed herein, to perform imaging, and reconstructs an image from the acquired intensity data. The monitor or display 1209 displays the reconstructed image, and the monitor or display 1209 may display other information about the imaging condition or about an object to be imaged. The monitor 1209 also provides a graphical user interface for a user to operate a system, for example when performing CT, MRI, or other imaging technique(s), including, but not limited to, controlling continuum robot devices/systems, and/or performing correction, adjustment, and/or smoothing technique(s). An operation signal is input from the operation unit (e.g., such as, but not limited to, a mouse device 1211, a keyboard 1210, a touch panel device, etc.) into the operation interface 1214 in the computer 1200′, and corresponding to the operation signal the computer 1200′ instructs the system (e.g., the system 1000, the systems/apparatuses of FIGS. 1-5, the systems/apparatuses of FIGS. 16-18, any other system/apparatus discussed herein,, etc.) to start or end the imaging, and/or to start or end continuum robot control(s) and/or performance of correction, adjustment, and/or smoothing technique(s). The camera or imaging device as aforementioned may have interfaces to communicate with the computers 1200, 1200′ to send and receive the status information and the control signals.

The present disclosure and/or one or more components of devices, systems, and storage mediums, and/or methods, thereof also may be used in conjunction with continuum robot devices, systems, methods, and/or storage mediums and/or with endoscope devices, systems, methods, and/or storage mediums. Such continuum robot devices, systems, methods, and/or storage mediums are disclosed in at least: U.S. Provisional Pat. App. No. 63/150,859, filed on Feb. 18, 2021, the disclosure of which is incorporated by reference herein in its entirety. Such endoscope devices, systems, methods, and/or storage mediums are disclosed in at least: U.S. patent application Ser. No. 17/565,319, filed on Dec. 29, 2021, the disclosure of which is incorporated by reference herein in its entirety; U.S. Pat. App. No. 63/132,320, filed on Dec. 30, 2020, the disclosure of which is incorporated by reference herein in its entirety; U.S. patent application Ser. No. 17/564,534, filed on Dec. 29, 2021, the disclosure of which is incorporated by reference herein in its entirety; and U.S. Pat. App. No. 63/131,485, filed Dec. 29, 2020, the disclosure of which is incorporated by reference herein in its entirety. Any of the features of the present disclosure may be used in combination with any of the features as discussed in U.S. Prov. Pat. App. No. 63/378,017, filed Sep. 30, 2022, the disclosure of which is incorporated by reference herein in its entirety, and/or any of the features as discussed in U.S. Prov. Pat. App. No. 63/377,983, filed Sep. 30, 2022, the disclosure of which is incorporated by reference herein in its entirety. Any of the features of the present disclosure may be used in combination with any of the features as discussed in U.S. Pat. Pub. No. 2023/0131269, published on Apr. 26, 2023, the disclosure of which is incorporated by reference herein in its entirety.

Although the disclosure herein has been described with reference to particular embodiments, it is to be understood that these embodiments are merely illustrative of the principles and applications of the present disclosure (and are not limited thereto), and the invention is not limited to the disclosed embodiments. It is therefore to be understood that numerous modifications may be made to the illustrative embodiments and that other arrangements may be devised without departing from the spirit and scope of the present disclosure. The scope of the following claims is to be accorded the broadest interpretation so as to encompass all such modifications, equivalent structures, and functions.

1. A continuum robot for performing correction, adjustment, and/or smoothing, the continuum robot comprising:
one or more processors that operate to:
instruct or command a distal bending section or portion of a catheter or a probe of the continuum robot such that the distal bending section or portion achieves, or is disposed at, a bending pose, position, or state, the catheter or probe of the continuum robot having a plurality of bending sections or portions and a base;
store or obtain the bending pose, position, or state of the distal bending section or portion and store or obtain a position or state of a motorized linear stage, of a rail moved by the motorized linear stage, and/or of a sensor that operates to move the catheter or probe of the continuum robot in a case where the one or more processors instruct or command forward motion, or a motion in a set or predetermined direction or directions, of the motorized linear stage and/or of the sensor;
generate a goal or target bending pose, position, or state for each corresponding section or portion of the catheter or probe from, or based on, the previous bending section or portion;
determine interpolated poses, positions, or states for each corresponding section or portion of the catheter or probe based on adjacent interpolated poses, positions or states such that all interpolated poses, positions, or states include the same or similar displacement vector between the adjacent interpolated poses, positions, or states based on an end-effector coordinate; and
instruct or command each of the sections or portions of the catheter or probe to move to or be disposed at the respective interpolated poses, positions, or states during the forward motion, or the motion in the set or predetermined direction, of the previous section(s) or portion(s) of the catheter or probe.

2. The continuum robot of claim 1, wherein the same or similar displacement vector between the adjacent interpolated poses, positions, or states is based on the end-effector coordinate from a view point of a distal end of that respective section or portion of the catheter or probe.

3. The continuum robot of claim 1, wherein one or more of the following:
(i) the plurality of bending sections or portions each include drive or driving wires that operate to bend a respective section or portion of the plurality of sections or portions, wherein the drive or driving wires are connected to the actuator so that the actuator operates to bend one or more of the plurality of bending sections or portions using the drive or driving wires; and/or
(ii) wherein a tip of the distal bending section or portion of the catheter or probe moves to a respective generated goal or target bending pose, position, or state at a same or constant change, speed, or velocity, or at substantially a same or constant change, speed, or velocity.

4. The continuum robot of claim 3, wherein, in a case where the plurality of bending sections or portions each include the drive or driving wires, the one or more processors further operate to one or more of the following:
(i) insert the continuum robot or one or more bending sections or portions of the catheter or probe of the continuum robot into an object, target, or sample, and/or control the rail and/or the motorized linear stage to insert the continuum robot or the one or more bending sections or portions of the catheter or probe of the continuum robot into the object, target, or sample;
(ii) use a Follow-The-Leader (FTL) process or algorithm to control each of the plurality of bending sections or portions of the catheter or probe such that the bending sections or portions of the catheter or probe follow a leader section or portion of the catheter or probe;
(iii) use a Follow-The-Leader (FTL) process or algorithm to control each of the plurality of bending sections or portions of the catheter or probe such that the bending sections or portions of the catheter or probe follow a leader section or portion of the catheter or probe, wherein the leader section or portion of the catheter or probe is or includes the distal bending section or portion and/or a tip of the distal bending section or portion of the catheter or probe;
(iv) use a Follow-The-Leader (FTL) process or algorithm to control each of the plurality of bending sections or portions of the catheter or probe such that the bending sections or portions of the catheter or probe follow a leader section or portion of the catheter or probe, wherein the leader section or portion of the catheter or probe is or includes the distal bending section or portion and/or a tip of the distal bending section or portion of the catheter or probe and wherein the bending sections or portions of the catheter or probe following the leader section or portion include at least a middle bending section or portion and a proximal bending section or portion;
(v) perform exploration by moving the distal bending section or portion or a tip of the distal bending section or portion to determine a final orientation, pose, position, or state to insert the continuum robot or one or more bending sections or portions of the catheter or probe of the continuum robot into an object, target, or sample at or along a position of or on the rail; and/or
(vi) use a Follow-The-Leader (FTL) process or algorithm to control each of the plurality of bending sections or portions of the catheter or probe such that the bending sections or portions of the catheter or probe follow a leader section or portion of the catheter or probe, and determine and/or store the bending pose, position, or state of the leader section or portion.

5. The continuum robot of claim 4, wherein, in a case where the continuum robot or the one or more bending sections or portions of the catheter or probe of the continuum robot are inserted into the object, target, or sample and where the Follow-The-Leader (FTL) process or algorithm is used, the one or more processors further operate to one or more of the following:
(i) perform the insertion into the object, target, or sample until a bifurcation of the object, target, or sample is reached or identified, and/or perform the insertion into the object, target, or sample simultaneously with exploration by moving the distal bending section or portion or a tip of the distal bending section or portion to determine a final orientation, pose, position, or state to insert the continuum robot or one or more bending sections or portions of the catheter or probe of the continuum robot into the object, target, or sample;
(ii) perform the insertion into the object, target, or sample until a bifurcation of the object, target, or sample is reached or identified, wherein the object, target, or sample is a lung or an airway of the lung;
(iii) in a case where a bifurcation of the object, target, or sample is reached, perform exploration by moving the leader section or portion, the distal bending section or portion, or a tip of the distal bending section or portion to determine an orientation, pose, position, or state with an endoscopic view to continue insertion of the continuum robot or the one or more bending sections or portions of the catheter or probe of the continuum robot along and into a path of the bifurcation until a following bifurcation of the object, target, or sample is reached or identified, and repeat the exploration for the following bifurcation until the leader section or portion, the distal bending section or portion, or the tip of the distal bending section or portion has reached the final orientation, pose, position, or state;
(iv) in a case where the bending sections or portions of the catheter or probe following the leader section or portion include at least a middle bending section or portion and a proximal bending section or portion, generate the goal or target bending pose, position, or state for at least the middle bending section or portion and the proximal bending section or portion; and/or
(v) in a case where the bending sections or portions of the catheter or probe following the leader section or portion include at least a middle bending section or portion and a proximal bending section or portion, generate the goal or target bending pose, position, or state for at least the middle bending section or portion and the proximal bending section or portion such that the goal or target bending pose, position, or state for at least the middle bending section or portion and the proximal bending section or portion are the same as, or are substantially the same as, the bending, pose, position, or state of one or more of the leader section or portion, the distal bending section or portion, and/or a tip of the leader section or portion and/or the distal bending section or portion in a case where the middle bending section or portion and the proximal bending section or portion reach the previous insertion position of the leader section or portion, the distal bending section or portion, and/or the tip of the leader section or portion.

6. The continuum robot of claim 4, wherein, in a case where the continuum robot or the one or more bending sections or portions of the catheter or probe of the continuum robot are inserted into the object, target, or sample and where the Follow-The-Leader (FTL) process or algorithm is used, the one or more processors further operate to one or more of the following:
(i) use an internal minimal insertion interval or length as an insertion control resolution to generate the interpolated poses, positions, or states; and/or
(ii) control all sections or portions of the catheter or probe smoothly using the FTL process or algorithm through the insertion by the internal minimal insertion interval or length.

7. The continuum robot of claim 4, wherein, in a case where the continuum robot or the one or more bending sections or portions of the catheter or probe of the continuum robot are inserted into the object, target, or sample and where the Follow-The-Leader (FTL) process or algorithm is used, the one or more processors further operate to one or more of the following:
(i) in a case where the bending sections or portions of the catheter or probe following the leader section or portion include at least a middle bending section or portion and a proximal bending section or portion, use the drive or driving wire positions to express the bending poses, positions, or states for one or more of: the bending pose, position, or state of the distal bending section or portion and/or the proximal or middle bending section(s) or portion(s); the goal or target bending pose, position, or state for each corresponding section or portion of the catheter or probe; and/or the interpolated poses, positions, or states for each corresponding section or portion of the catheter or probe;
(ii) convert current and goal bending poses, positions, or states to corresponding drive or driving wire positions for each section or portion of the catheter or probe;
(iii) convert current and goal bending poses, positions, or states to corresponding drive or driving wire positions for each section or portion of the catheter or probe, and interpolate the drive or driving wire positions between the current and goal drive or driving wire positions;
(iv) convert current and goal bending poses, positions, or states to corresponding drive or driving wire positions for each section or portion of the catheter or probe, and store the drive or driving wire positions of the distal bending section or portion and/or update a lookup table with the drive or driving wire positions;
(v) in a case where the bending sections or portions of the catheter or probe following the leader section or portion include at least a middle bending section or portion and a proximal bending section or portion, convert current and goal bending poses, positions, or states to corresponding drive or driving wire positions for each section or portion of the catheter or probe, determine the drive or driving wire positions of the distal bending section or portion, the middle bending section or portion, and/or the proximal bending section or portion, and compute the goal drive or driving wire positions for at least a middle section or portion and a proximal section or portion of the catheter or probe out of, or based on, the drive or driving wire positions of the distal bending section or portion, or select one of the distal bending section or portion, the middle bending section or portion, and the proximal bending section or portion and compute the goal drive or driving wire positions for the other two sections or portions of the catheter or probe out of, or based on, the selected one of the distal bending section or portion, the middle bending section or portion, and/or the proximal bending section or portion;
(vi) in a case where the bending sections or portions of the catheter or probe following the leader section or portion include at least a middle bending section or portion and a proximal bending section or portion, use a cross-sectional position difference of a predetermined or set number of the drive or driving wires between the distal and middle sections such that the positions of the drive or driving wires for a bending pose, position, or state are converted into positions for another or a different predetermined or set number of the drive or driving wires at and/or using the difference cross-sectional position; and/or
(vii) in a case where the bending sections or portions of the catheter or probe following the leader section or portion include at least a middle bending section or portion and a proximal bending section or portion, create linear interpolation of the drive or driving wire positions between the current and goal drive or driving wire positions of the middle bending section for every minimal insertion interval, and/or take a difference between the current and goal drive or driving wire positions mapped to a target stage position, create the linear interpolation of the difference, and add the linear interpolation to the corresponding values at every minimal insertion level and/or add a portion of a total wire position different to each point along an interpolated range so that current bending poses, positions, or states and drive or driving wire positions gradually aim toward the goal or final target pose, position, or state while maintaining and ensuring that each section or portion of the catheter or probe has minimal or reduced interaction with the object, target, or sample.

8. The continuum robot of claim 4, wherein, in a case where the continuum robot or the one or more bending sections or portions of the catheter or probe of the continuum robot are inserted into the object, target, or sample and where the Follow-The-Leader (FTL) process or algorithm is used, the one or more processors further operate to one or more of the following:
(i) convert current and goal bending poses, positions, or states to corresponding drive or driving wire positions for each section or portion of the catheter or probe, and interpolate the drive or driving wire positions between the current and goal drive or driving wire positions such that each section or portion bends at a constant rate of change, speed, and/or velocity through the insertion; and/or
(ii) control each section or portion to approach from the current pose, position, or state with the constant rate of change, speed, and/or velocity such that the insertion routes along a shorter path and/or such that the continuum robot operates to reduce or avoid an interaction to or with any predetermined portion of the target, object, or sample.

9. The continuum robot of claim 3, wherein, in a case where the plurality of bending sections or portions each include the drive or driving wires, the one or more processors further operate to one or more of the following:
generate the interpolated poses, positions, or states by interpolating positions or states of the drive or driving wires between the respective goal or target poses, positions, or states and the respective current poses, positions, or states;
take a difference in drive or driving wire position between a value stored in a table and a new value; and
disperse an adjustment, correction, and/or smoothing along an entire length of travel from a current pose, position, or state of the bending sections or portions by:
(i) calculating a number of steps by multiplying a length of a section or portion and a table resolution;
(ii) interpolating the value from the table into equal portions where the number of the equal portions equals the calculated number of steps, starting with the total value and ending at zero; and
(iii) working backwards from the position in the table where the new value is getting stored, and adding the interpolated pose, position, or state value to the corresponding value stored in the table, wherein StagePosition[0] is the position where the new value is to be stored, InterpolatedPosition[0] is the full amount of the wire pose, position, or state difference, StagePosition[−i] is the pose, position, or state where the smoothing begins, and InterpolatedPosition[i] has zero wire pose, position, or state difference.

10. A method for performing correction, adjustment, and/or smoothing for a continuum robot, the method comprising:
instructing or commanding a distal bending section or portion of a catheter or a probe of the continuum robot such that the distal bending section or portion achieves, or is disposed at, a bending pose, position, or state, the catheter or probe of the continuum robot having a plurality of bending sections or portions and a base;
storing or obtaining the bending pose, position, or state of the distal bending section or portion and store or obtain a position or state of a motorized linear stage and/or of a sensor that operates to move the catheter or probe of the continuum robot in a case where the one or more processors instruct or command forward motion, or a motion in a set or predetermined direction or directions, of the motorized linear stage and/or of the sensor;
generating a goal or target bending pose, position, or state for each corresponding section or portion of the catheter or probe from, or based on, the previous bending section or portion;
determining interpolated poses, positions, or states for each corresponding section or portion of the catheter or probe based on adjacent interpolated poses, positions or states such that all interpolated poses, positions, or states include the same or similar displacement vector between the adjacent interpolated poses, positions, or states based on an end-effector coordinate; and
instructing or commanding each of the sections or portions of the catheter or probe to move to or be disposed at the respective interpolated poses, positions, or states during the forward motion, or the motion in the set or predetermined direction, of the previous section(s) or portion(s) of the catheter or probe.

11. The method of claim 10, wherein the same or similar displacement vector between the adjacent interpolated poses, positions, or states is based on the end-effector coordinate from a view point of a distal end of that respective section or portion of the catheter or probe.

12. The method of claim 10, wherein one or more of the following:
(i) the plurality of bending sections or portions each include drive or driving wires that operate to bend a respective section or portion of the plurality of sections or portions, wherein the drive or driving wires are connected to the actuator so that the actuator operates to bend one or more of the plurality of bending sections or portions using the drive or driving wires; and/or
(ii) wherein a tip of the distal bending section or portion of the catheter or probe moves to a respective generated goal or target bending pose, position, or state at a same or constant change, speed, or velocity, or at substantially a same or constant change, speed, or velocity.

13. The method of claim 12, further comprising, in a case where the plurality of bending sections or portions each include the drive or driving wires, one or more of the following:
(i) inserting the continuum robot or one or more bending sections or portions of the catheter or probe of the continuum robot into an object, target, or sample, and/or controlling the rail and/or the motorized linear stage to insert the continuum robot or the one or more bending sections or portions of the catheter or probe of the continuum robot into the object, target, or sample;
(ii) using a Follow-The-Leader (FTL) process or algorithm to control each of the plurality of bending sections or portions of the catheter or probe such that the bending sections or portions of the catheter or probe follow a leader section or portion of the catheter or probe;
(iii) using a Follow-The-Leader (FTL) process or algorithm to control each of the plurality of bending sections or portions of the catheter or probe such that the bending sections or portions of the catheter or probe follow a leader section or portion of the catheter or probe, wherein the leader section or portion of the catheter or probe is or includes the distal bending section or portion and/or a tip of the distal bending section or portion of the catheter or probe;
(iv) using a Follow-The-Leader (FTL) process or algorithm to control each of the plurality of bending sections or portions of the catheter or probe such that the bending sections or portions of the catheter or probe follow a leader section or portion of the catheter or probe, wherein the leader section or portion of the catheter or probe is or includes the distal bending section or portion and/or a tip of the distal bending section or portion of the catheter or probe and wherein the bending sections or portions of the catheter or probe following the leader section or portion include at least a middle bending section or portion and a proximal bending section or portion;
(v) performing exploration by moving the distal bending section or portion or a tip of the distal bending section or portion to determine a final orientation, pose, position, or state to insert the continuum robot or one or more bending sections or portions of the catheter or probe of the continuum robot into an object, target, or sample at or along a position of or on the rail; and/or
(vi) using a Follow-The-Leader (FTL) process or algorithm to control each of the plurality of bending sections or portions of the catheter or probe such that the bending sections or portions of the catheter or probe follow a leader section or portion of the catheter or probe, and determine and/or store the bending pose, position, or state of the leader section or portion.

14. The method of claim 13, further comprising, in a case where the continuum robot or the one or more bending sections or portions of the catheter or probe of the continuum robot are inserted into the object, target, or sample and where the Follow-The-Leader (FTL) process or algorithm is used, one or more of the following:
(i) performing the insertion into the object, target, or sample until a bifurcation of the object, target, or sample is reached or identified, and/or performing the insertion into the object, target, or sample simultaneously with exploration by moving the distal bending section or portion or a tip of the distal bending section or portion to determine a final orientation, pose, position, or state to insert the continuum robot or one or more bending sections or portions of the catheter or probe of the continuum robot into the object, target, or sample;
(ii) performing the insertion into the object, target, or sample until a bifurcation of the object, target, or sample is reached or identified, wherein the object, target, or sample is a lung or an airway of the lung;
(iii) in a case where a bifurcation of the object, target, or sample is reached, performing exploration by moving the leader section or portion, the distal bending section or portion, or a tip of the distal bending section or portion to determine an orientation, pose, position, or state with an endoscopic view to continue insertion of the continuum robot or the one or more bending sections or portions of the catheter or probe of the continuum robot along and into a path of the bifurcation until a following bifurcation of the object, target, or sample is reached or identified, and repeating the exploration for the following bifurcation until the leader section or portion, the distal bending section or portion, or the tip of the distal bending section or portion has reached the final orientation, pose, position, or state;
(iv) in a case where the bending sections or portions of the catheter or probe following the leader section or portion include at least a middle bending section or portion and a proximal bending section or portion, generating the goal or target bending pose, position, or state for at least the middle bending section or portion and the proximal bending section or portion; and/or
(v) in a case where the bending sections or portions of the catheter or probe following the leader section or portion include at least a middle bending section or portion and a proximal bending section or portion, generating the goal or target bending pose, position, or state for at least the middle bending section or portion and the proximal bending section or portion such that the goal or target bending pose, position, or state for at least the middle bending section or portion and the proximal bending section or portion are the same as, or are substantially the same as, the bending, pose, position, or state of one or more of the leader section or portion, the distal bending section or portion, and/or a tip of the leader section or portion and/or the distal bending section or portion in a case where the middle bending section or portion and the proximal bending section or portion reach the previous insertion position of the leader section or portion, the distal bending section or portion, and/or the tip of the leader section or portion.

15. The method of claim 13, further comprising, in a case where the continuum robot or the one or more bending sections or portions of the catheter or probe of the continuum robot are inserted into the object, target, or sample and where the Follow-The-Leader (FTL) process or algorithm is used, one or more of the following:
(i) using an internal minimal insertion interval or length as an insertion control resolution to generate the interpolated poses, positions, or states; and/or
(ii) controlling all sections or portions of the catheter or probe smoothly using the FTL process or algorithm through the insertion by the internal minimal insertion interval or length.

16. The method of claim 13, further comprising, in a case where the continuum robot or the one or more bending sections or portions of the catheter or probe of the continuum robot are inserted into the object, target, or sample and where the Follow-The-Leader (FTL) process or algorithm is used, one or more of the following:
(i) in a case where the bending sections or portions of the catheter or probe following the leader section or portion include at least a middle bending section or portion and a proximal bending section or portion, using the drive or driving wire positions to express the bending poses, positions, or states for one or more of: the bending pose, position, or state of the distal bending section or portion and/or the proximal or middle bending section(s) or portion(s); the goal or target bending pose, position, or state for each corresponding section or portion of the catheter or probe;
and/or the interpolated poses, positions, or states for each corresponding section or portion of the catheter or probe;
(ii) converting current and goal bending poses, positions, or states to corresponding drive or driving wire positions for each section or portion of the catheter or probe;
(iii) converting current and goal bending poses, positions, or states to corresponding drive or driving wire positions for each section or portion of the catheter or probe, and interpolating the drive or driving wire positions between the current and goal drive or driving wire positions;
(iv) converting current and goal bending poses, positions, or states to corresponding drive or driving wire positions for each section or portion of the catheter or probe, and storing the drive or driving wire positions of the distal bending section or portion and/or updating a lookup table with the drive or driving wire positions;
(v) in a case where the bending sections or portions of the catheter or probe following the leader section or portion include at least a middle bending section or portion and a proximal bending section or portion, converting current and goal bending poses, positions, or states to corresponding drive or driving wire positions for each section or portion of the catheter or probe, determining the drive or driving wire positions of the distal bending section or portion, the middle bending section or portion, and/or the proximal bending section or portion, and computing the goal drive or driving wire positions for at least a middle section or portion and a proximal section or portion of the catheter or probe out of, or based on, the drive or driving wire positions of the distal bending section or portion, or selecting one of the distal bending section or portion, the middle bending section or portion, and the proximal bending section or portion and computing the goal drive or driving wire positions for the other two sections or portions of the catheter or probe out of, or based on, the selected one of the distal bending section or portion, the middle bending section or portion, and/or the proximal bending section or portion;
(vi) in a case where the bending sections or portions of the catheter or probe following the leader section or portion include at least a middle bending section or portion and a proximal bending section or portion, using a cross-sectional position difference of a predetermined or set number of the drive or driving wires between the distal and middle sections such that the positions of the drive or driving wires for a bending pose, position, or state are converted into positions for another or a different predetermined or set number of the drive or driving wires at and/or using the difference cross-sectional position; and/or
(vii) in a case where the bending sections or portions of the catheter or probe following the leader section or portion include at least a middle bending section or portion and a proximal bending section or portion, creating linear interpolation of the drive or driving wire positions between the current and goal drive or driving wire positions of the middle bending section for every minimal insertion interval, and/or taking a difference between the current and goal drive or driving wire positions mapped to a target stage position, creating the linear interpolation of the difference, and adding the linear interpolation to the corresponding values at every minimal insertion level and/or adding a portion of a total wire position different to each point along an interpolated range so that current bending poses, positions, or states and drive or driving wire positions gradually aim toward the goal or final target pose, position, or state while maintaining and ensuring that each section or portion of the catheter or probe has minimal or reduced interaction with the object, target, or sample.

17. The method of claim 13, further comprising, in a case where the continuum robot or the one or more bending sections or portions of the catheter or probe of the continuum robot are inserted into the object, target, or sample and where the Follow-The-Leader (FTL) process or algorithm is used, one or more of the following:
(i) converting current and goal bending poses, positions, or states to corresponding drive or driving wire positions for each section or portion of the catheter or probe, and interpolating the drive or driving wire positions between the current and goal drive or driving wire positions such that each section or portion bends at a constant rate of change, speed, and/or velocity through the insertion; and/or
(ii) controlling each section or portion to approach from the current pose, position, or state with the constant rate of change, speed, and/or velocity such that the insertion routes along a shorter path and/or such that the continuum robot operates to reduce or avoid an interaction to or with any predetermined portion of the target, object, or sample.

18. A non-transitory computer-readable storage medium storing at least one program for causing a computer to execute the method of claim 10.

19-22. (canceled)

23. The continuum robot of claim 1, wherein the one or more processors further operate to:
generate the interpolated poses, positions, or states for each of the sections or portions of the catheter or probe between the respective goal or target bending pose, position, or state and a respective current bending pose, position, or state of each of the sections or portions of the catheter or probe, wherein the interpolated poses, positions, or states are generated such that an orientation vector of the interpolated poses, positions, or states is on a plane that an orientation vector of the respective goal or target bending pose, position, or state and an orientation vector of a respective current bending pose, position, or state create or define.

24. The continuum robot of claim 23, wherein one or more of the following:
(i) the distal bending section or portion is the most distal bending section or portion, and the most distal bending section or portion is commanded or instructed automatically or based on an input of a user of the continuum robot in a case where the motorized linear stage and/or another component that operates to move the probe or catheter is stable or stationary;
(ii) the plurality of bending sections or portions includes the distal or most distal bending portion or section and the rest of the plurality of the bending sections or portions;
(iii) the one or more processors further operate to instruct or command the forward motion, or the motion in the set or predetermined direction, of the motorized linear stage and/or of the sensor automatically or based on an input of a user of the continuum robot; and/or
(iv) the plane is created or defined based on a base coordinate system or based on a system substantially close to the base coordinate system.

25. The continuum robot of claim 1, further comprising:
an actuator that operates to bend the plurality of the bending sections or portions independently and the base; and
the motorized linear stage and/or the sensor that operates to move the continuum robot forward and backward, and/or in the predetermined or set direction or directions,
wherein the one or more processors operate to control the actuator and the motorized linear stage and/or the sensor.

26. The continuum robot of claim 25, further comprising a user interface of or disposed on the base, or disposed remotely from the base, the user interface operating to receive an input from a user of the continuum robot to move one or more of the plurality of bending sections or portions and/or the motorized linear stage and/or the sensor, wherein the one or more processors further operate to receive the input from the user interface, and the one or more processors and/or the user interface operate to use a base coordinate system.

27. The continuum robot of claim 25, wherein the plurality of bending sections or portions each include drive or driving wires that operate to bend a respective section or portion of the plurality of sections or portions, wherein the drive or driving wires are connected to the actuator so that the actuator operates to bend one or more of the plurality of bending sections or portions using the drive or driving wires.

28. The continuum robot of claim 27, wherein the one or more processors further operate to one or more of the following:
generate the interpolated poses, positions, or states by interpolating positions or states of the driving wires between the respective goal or target poses, positions, or states and the respective current poses, positions, or states;
take a difference in drive or driving wire position between a value stored in a table and a new value; and
disperse an adjustment, correction, and/or smoothing along an entire length of travel from a current pose, position, or state of the bending sections or portions by:
(iv) calculating a number of steps by multiplying a length of a section or portion and a table resolution;
(v) interpolating the value from the table into equal portions where the number of the equal portions equals the calculated number of steps, starting with the total value and ending at zero; and
(vi) working backwards from the position in the table where the new value is getting stored, and adding the interpolated pose, position, or state value to the corresponding value stored in the table, wherein StagePosition[0] is the position where the new value is to be stored, InterpolatedPosition[0] is the full amount of the wire pose, position, or state difference, StagePosition[−i] is the pose, position, or state where the smoothing begins, and InterpolatedPosition[i] has zero wire pose, position, or state difference.

29. The continuum robot of claim 28, wherein the adjustment, correction, and/or smoothing occurs such that any intermediate orientations and/or poses, positions, or states of one or more of the plurality of bending sections or portions is guided towards respective desired, predetermined, or set orientations and/or poses, positions, or states.

30. The continuum robot of claim 29, further including a display to display the adjusted, corrected, or smoothed path of the continuum robot.

31. The continuum robot i claim 1, wherein one or more of the following:
(i) the continuum robot further comprises an operational controller or joystick that operates to issue or input one or more commands or instructions as an input to the one or more processors, the input including an instruction or command to move one or more of the plurality of bending sections or portions and/or the motorized linear stage and/or the sensor;
(ii) the continuum robot further includes a display to display one or more images taken by the continuum robot; and/or
(iii) the continuum robot further comprises an operational controller or joystick that operates to issue or input one or more commands or instructions to the one or more processors, the input including an instruction or command to move one or more of the plurality of bending sections or portions and/or the motorized linear stage and/or the sensor, and the operational controller or joystick operates to be controlled by a user of the continuum robot.

32. The continuum robot of claim 1, wherein the catheter or probe of the continuum robot is a steerable catheter or probe including the plurality of bending sections or portions and including an endoscope camera, wherein the one or more processors further operate to receive one or more endoscopic images from the endoscope camera, and wherein the continuum robot further comprises a display that operates to display the one or more endoscopic images.

33. The method of claim 10, further comprising:
generating the interpolated poses or positions for each of the sections or portions of the catheter or probe between the respective goal or target bending pose, position, or state and a respective current bending pose, position, or state of each of the sections or portions of the catheter or probe, wherein the interpolated poses, positions, or states are generated such that an orientation vector of the interpolated poses, positions, or states is on a plane that an orientation vector of the respective goal or target bending pose, position, or state and an orientation vector of a respective current bending pose, position, or state create or define.

34-51. (canceled)
